(19) United States
(12) Patent Application Publication
(10) Pub. No.: US 2008/0090765 A1
(43) Pub. Date: Apr. 17, 2008

(54) Compositions for modulating growth of embryonic and adult kidney tissue and uses for treating kidney damage

(75) Inventors: Kai Schmidt-Ott, Berlin (DE); Jonathan Barasch, New York, NY (US); Jun Yang, New York, NY (US)

Correspondence Address: WilmerHale/Columbia University, 399 PARK AVENUE, NEW YORK, NY, 10022, US

(73) Assignee: THE TRUSTEES OF COLUMBIA UNIVERSITY IN THE CITY OF NEW YORK, New York, NY (US)

(21) Appl. No.: 11/807,250

(22) Filed: May 25, 2007

Related U.S. Application Data
(60) Provisional application No. 60/808,491, filed on May 25, 2006.

(51) Int. Cl. A61K 38/17; A61P 43/00

(52) U.S. Cl. 514/12

(57) ABSTRACT

The invention is directed to compositions comprising two or more compounds selected from the group consisting of stem cell factor, cytokine like factor-1, CXCL14, FRAS1, neuropeptide Y, Semaphorin 3C, Cyr 61, USAG-1, IGF-BP2, WNT 6, WNT 9B, SHH, BMP-7, kit ligand, SOSTDC1, semaphorin 4D, NME3, laminin gamma 2, laminin alpha 5, laminin gamma 1, collagen triple helix repeat containing 1, nephronectin, collagen XVIII and laminin alpha 1, and methods of using the compositions to modulate the growth of embryonic or adult kidney tissue or to treat kidney damage in a mammal. The invention is also related to a kit for treating kidney damage.

This application claims priority to U.S. Provisional Application No. 60/808,491, filed May 25, 2006, which is hereby incorporated by reference in its entirety.

The invention disclosed herein was made with U.S. Government support under National Institutes of Health Grant Numbers NIH DK 55388 and NIH DK58872. Accordingly, the U.S. Government may have certain rights in this invention.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described herein.

### BACKGROUND OF THE INVENTION

Most organs in the body, including lung, pancreas, exocrine glands and kidney, consist of a cell type called epithelia. These cells function to transport substances in a vectorial fashion, allowing directed absorption of nutrients, salts and water and directed release of newly synthesized proteins. The kidney is an epithelial organ that moves water, salts, small organic chemicals and small hormones between the blood and the forming urine, and this directed absorption and secretion achieves consistent concentrations of these substances throughout the body. In the kidney, the partitioning of blood and urinary components starts in a long epithelial tubule called the nephron and human kidneys have between 0.5-1.5 million nephrons.

The development of the nephron is different from the formation of most other epithelial tubules. Whereas most epithelial tubules (the lung, pancreas, exocrine glands, the kidney's collecting ducts) derive from epithelial tubules and sheets that were generated much earlier in development, the epithelium of the nephron derives directly from a second cell type called mesenchyme. The conversion process is well described by observations in the microscope and some genes are known to be important for the conversion process, for example the Wnt family of secreted growth factors and the transcription factors Pax-2 and WT-1.

Nephrons are generated when branches of the ureteric bud (the future collecting duct) contact mesenchymal cells and induce them to undergo differentiation to epithium. The ureteric bud (UB) is essential for two separate events in the metanephric mesenchyme: it produces factors that permit the survival of progenitor cells, and it produces factors that convert these cells into epithelial tubules.

To uncover the basic mechanisms of kidney formation, it is important to find as many of these ureteric bud factors as possible, and then to explore their effects in the embryonic kidney. The activity of these molecules is measured by their ability to generate progenitors, convert these progenitors into epithelial cells, tubules and fully formed nephrons. Methods to replicate the formation of the nephron are few.

In addition to identifying regulators of epithelial formation using embryonic tissues, it is also important to test the regulators in models of epithelial re-formation in adult tissue, especially in settings of acute tubular necrosis (ATN), a condition that is common in hospitalized patients, especially in intensive care units (For review see Esson and Schrier, Ann Intern Med 137:744-752 (2002)). Presently, there are no known specific therapies to treat ATN. Despite advances in dialysis-based treatments, the mortality rate from ATN has remained at 50%-80% over the past four decades.

### SUMMARY OF THE INVENTION

In one aspect, compositions and methods are provided for modulating the growth of metanephric tissue.

In one aspect, a composition is provided for modulating growth of metanephric tissue, the composition consisting essentially of two or more compounds selected from the group consisting of stem cell factor, cytokine like factor-1, CXCL14, FRAS1, neuropeptide Y, semaphorin 3C, Cyr 61, USAG-1, IGFBP2, WNT 6, WNT 9B, SHH, BMP7, kit ligand, SOSTDC1, semaphorin 4D, NME3, laminin gamma 2, laminin alpha 5, laminin gamma 1, collagen triple helix repeat containing 1, nephronectin, collagen XVIII, and laminin alpha 1.

In one aspect, a composition is provided for modulating growth of metanephric tissue, the composition consisting essentially of two or more compounds selected from the group consisting of stem cell factor, cytokine like factor-1, CXCL14, neuropeptide Y, semaphorin 3C, Cyr 61, IGFBP2, semaphorin 4D, NME3, and collagen triple helix repeat containing 1.

In one aspect, a composition is provided for modulating growth of metanephric tissue, the composition consisting essentially of WNT 6 and one or more compounds selected from the group consisting of stem cell factor, cytokine like factor-1, CXCL14, FRAS1, neuropeptide Y, semaphorin 3C, Cyr 61, USAG-1, IGFBP2, WNT 9B, SHH, BMP7, kit ligand, SOSTDC1, semaphorin 4D, NME3, laminin gamma 2, laminin alpha 5, laminin gamma 1, collagen triple helix repeat containing 1, nephronectin, collagen XVIII and laminin alpha 1.

In one aspect, a composition is provided for modulating growth of metanephric tissue, the composition consisting essentially of WNT 9B and one or more compounds selected from the group consisting of stem cell factor, cytokine like factor-1, CXCL14, FRAS1, neuropeptide Y, semaphorin 3C, Cyr 61, USAG-1, IGFBP2, SHH, BMP7, kit ligand, SOSTDC1, semaphorin 4D, NME3, laminin gamma 2, laminin alpha 5, laminin gamma 1, collagen triple helix repeat containing 1, nephronectin, collagen XVIII and laminin alpha 1.

In one aspect, a composition is provided for modulating growth of metanephric tissue, the composition consisting essentially of cytokine like factor-1 (CLF-1) and BMP7.

In one aspect, a composition is provided for modulating growth of metanephric tissue, the composition consisting essentially of cytokine like factor-1 (CLF-1) and cardiotrophin like cytokine (CLC).

In one aspect, a method is provided for modulating growth of embryonic or adult kidney tissue, the method comprising contacting the embryonic or adult kidney tissue with an effective amount of cytokine like factor-1 (CLF-1).

In one aspect, a method is provided for modulating growth of embryonic or adult kidney tissue, the method comprising contacting the embryonic or adult kidney tissue with an effective amount of stem cell factor.

In one aspect, a method is provided for modulating growth of embryonic or adult kidney tissue, the method comprising contacting the embryonic or adult kidney tissue with an effective amount of any one of the compositions provided by the invention.

In one aspect, a method is provided for modulating conversion of metanephric tissue to nephron epithelium, the method comprising contacting the embryonic or adult kidney tissue with an effective amount of cytokine like factor-1 (CLF-1).

In one aspect, a method is provided for modulating conversion of metanephric tissue to nephron epithelium, the method comprising contacting the embryonic or adult kidney tissue with an effective amount of stem cell factor.

In one aspect, a method is provided for modulating conversion of metanephric tissue to nephron epithelium, the method comprising contacting the metanephric tissue with an effective amount of any one of the compositions provided by the invention.

In one embodiment, the tissue comprises metanephric stem cells, renal stem cells, or both. In one embodiment, the tissue is in the kidney of a subject. In one embodiment, the tissue is isolated from an embryonic kidney, fetal kidney, developing kidney, or adult kidney. In one embodiment, the tissue is contained within an embryonic kidney, fetal kidney, developing kidney, or adult kidney, wherein the kidney is in an organ culture. In one embodiment, the tissue is transplanted into a subject. In one embodiment, the kidney is transplanted into a subject.

In other embodiments, the subject is a human, mouse, rabbit, monkey, rat, bovine, pig, sheep, goat, cow or dog. In one embodiment, the subject is suffering from kidney disease or kidney damage. In one embodiment, the subject is suffering from or undergoing acute tubular necrosis, acute renal failure, transplant, diabetes, infection, surgery, ischemia, muscle damage, liver disease, blood transfusion, exposure to nephrotoxic medication or agents, or any combination thereof.

In one embodiment, the tissue is contacted by the composition for at least 24 hours. In one embodiment, the effective amount of the composition comprises from about 50 nanograms to about 50 micrograms.

In one aspect, a method is provided for treating damaged kidney tissue, the method comprising administering to a subject an effective amount of any one of the compositions provided by the invention. In one embodiment, the subject is suffering from kidney damage resulting from acute tubular necrosis, acute renal failure, transplant, diabetes, infection, surgery, ischemia, muscle damage, liver disease, blood transfusion, exposure to nephrotoxic medication or agents, or any combination thereof. In one embodiment, the administering comprises intralesional, intraperitoneal, intramuscular or intravenous injection; infusion; liposome-mediated delivery; or topical, nasal, oral, ocular or otic delivery. In one embodiment, the administering is through the renal artery. In one embodiment, the effective amount of the composition comprises from about 50 nanograms to about 50 micrograms.

In one aspect a kit is provided for treating kidney damage comprising any one of the compositions of the invention in an effective amount to modulate the growth of embryonic or adult kidney tissue. In one embodiment, the kit is used during dialysis. In another embodiment, the kit is used with a drug delivery pump. In one embodiment, the pump is connected by a catheter to the renal artery. In another embodiment, the pump is implanted into a subject.

In another embodiment, the kit is used to prepare embryonic kidney cells, adult kidney cells, or a combination thereof for use in a bioartificial hemofiltration device. In one embodiment, the bioartificial hemofiltration device is implanted into a subject.

In one aspect, a method is provided for protecting a kidney from damage, the method comprising contacting the kidney with an effective amount of any one of the compositions provided by the invention.

In one aspect, a method is provided for stimulating nephron repair, the method comprising contacting the nephron in need of repair with an effective amount of a composition provided by the invention.

In some embodiments, the administration of the composition of the invention may be effected by intralesional, intraperitoneal, intramuscular or intravenous injection; by infusion; or may involve liposome-mediated delivery; or topical, nasal, oral, anal, ocular or otic delivery.

In other embodiments, administration of the inhibitor may comprise daily, weekly, monthly or hourly administration, the precise frequency being subject to various variables such as age and condition of the subject, amount to be administered, half-life of the agent in the subject, area of the subject to which administration is desired and the like.

In other embodiments, therapeutically effective amount of the inhibitor may include dosages which take into account the size and weight of the subject, the age of the subject, the severity of the obesity-related symptoms, the method of delivery of the agent and the history of the symptoms in the subject.

### BRIEF DESCRIPTION OF THE FIGURES

FIGS. 1A-1B: FIG. 1A. Isolation of FGF-2 from the conditioned media of the ureteric bud cell lines. Liters of conditioned media were collected and fractionated by heparin sepharose chromatography. The growth effects seen in fractions 49-55 were attributed to FGF-2. FIG. 1B. The mesenchymal stem cells of the kidney, the progenitors of the kidneys nephrons, are not capable of survival without the ureteric bud and undergo apoptosis (left), but in the presence of FGF-2 or FGF-9 or other FGFs the tissue survived and grew (right), but in no case was able to be converted into epithelia.

FIG. 2: Mesenchymal stem cells were maintained in culture for 4 days with FGF-2 (bFGF) and then were treated with an inductive tissue. The appearance of epithelia shown in Panels A and B demonstrates that FGF-2 maintains renal progenitors.

FIG. 3: Metanephric mesenchymal stem cells were rescued from apoptosis by FGF (Panel A). However, these cells did not produce epithelia. When treated with LIF, robust conversion was found (Panels B, C). Inspection of the tissue showed the presence of fully segmented nephrons.

FIG. 4: FGF+LIF induces polarized epithelia. The basal distribution of Collagen IV and the apical location of E-Cadherin demonstrates polarized epithelia. Only the combination of FGF and LIF was observed to induce epithelia.

FIG. 5: The entire nephron is induced by LIF including distal tubule (E-cadherin+), proximal tubules (lotus lectin+) and glomeruli (PNA+).

FIG. 6: Metanephric mesenchyme cultured with FGF produce clusters of metanephric stem cells that can be detected as PAX2+ Wnt4+ and WT1+ but tenascin negative (a marker for renal stroma). Note that stromal cells surround the clusters of metanephric mesenchyme.

FIGS. 7A-7E: The addition of LIF to the metanephric mesenchyme resulted in the phosphorylation of STAT3 (FIG. 7A), the onset of expression of SOC genes (FIG. 7B) and the uptake of BrDU signifying cell proliferation FIGS. 7C and 7D). FIG. 7E shows the differentiation of the clusters of mesenchymal stem cells that have been extracted from the embryonic kidney. These clusters do not contain stromal elements and are pure stem cells.

FIG. 8: The isolated metanephric mesenchymal cluster of stem cells produces glomeruli and tubules when incubated with LIF (PNA and Podocalyxin=glomeruli, E-cadherin is typical of distal nephrons). The lower panel shows the result of application of media conditioned by stromal cells to the clusters that have been treated with LIF. While E-cadherin+epithelia are generated, there are no PNA or podocalyxin glomeruli.

FIG. 9: Purification of NGAL from liters of conditioned media from the UB cell line. Shown is a silver stain of the final purified protein.

FIG. 10: NGAL targets cells at the periphery of the kidney. By using BF2-GFP transgenic mice, at least some of the labeling is detected in the kidney stroma (BF-2-GFP).

FIGS. 11A-11G: A fluorescent probe was used to detect cell iron. 5′IRE-YFP acts a suppressor (compare FIGS. 11A and 11B) but then increases in intensity with iron dosing (compare FIGS. 11C and 11D), but 3′IRE-YFP decreases in activity with iron loading (compare FIGS. 11E and F). The signal can be followed by FACS (FIG. 11G) and fluorescence (FIGS. 11A-11F).

FIG. 12: A fluorescent probe was used to detect iron in a single cell. 5′IRE-YFP increases in intensity with iron dosing, but control probe shows no changes. This is a single cell measurement by time-lapse cinematography.

FIG. 13: Urinary NGAL is dose dependent on the length of the ischemic event. The greater the dose of ischemia, the earlier a greater amount of NGAL appears in the urine.

FIG. 14: Infusion of fluorescent NGAL targets the kidney. The protein is filtered and then taken up by the proximal tubule into vesicles.

FIG. 15: Rescue of ischemic kidney (ATN) by injecting NGAL (10-100 ug/mouse). The Ngal is a complex of protein (Ngal)+siderophore (enterochelin)+Fe. This figure shows the preservation of structure and the cortico-medullary junction in the Ngal treated animal. This figure also shows the complete loss of nuclei (necrosis) of the proximal tubule.

FIG. 16: Discovery of the molecules expressed by the ureteric bud. The tips were cut off of branched E12.5 mouse and E14.5 rat ureteric buds. One thousand tip segments and stalk segments were collected and then microarrays were performed. The result is the collection of all genes in the ureteric bud that stimulate the growth and development of the kidney. (See Schmidt-Ott K M et al., J Am Soc Nephrol. 2005 July; 16(7):1993-2002).

FIG. 17: Discovery of secreted growth factors from the ureteric bud. These in situ hybridizations confirm the gene chip identifications of novel secreted growth factors. Note that ectodin is another name for USAG and Kitl is stem cell factor.

FIG. 18: Discovery of the inductive effects of CLF paired with its physiological ligand CLC. Also the family member of CLC/CLF is CNTF (Ciliary Neurotrophic Factor) and its inductive effects are shown. The lobulation of the mesenchyme is a marker of a dense field of tubules.

FIGS. 19A-19H: Discovery of SCF in the kidney is demonstrated by in situ hybridization (FIGS. 19A-19C). SCF locates in the ureteric bud (both tips and stalks). FIGS. 19D-19H show that SCF expands the kidney and increases the number of glomeruli 130%, whereas an inhibitor of the SCF receptor, STI, causes retarded kidney growth and limits glomerulogenesis to 80%.

FIGS. 20A-20K: Rat metanephric mesenchymes recapitulate differentiation of kidney epithelia in vivo under defined culture conditions. Metanephric mesenchymes cultured in basal media undergo apoptosis (A). Addition of FGF-2 and TGF-α to the culture media induces survival of clusters of progenitors (B). These aggregates express Wnt-4 as detected by in situ hybridization (C). After continued culture with FGF-2 and TGF-α, mesenchymes degenerate without differentiating into epithelia (D). NHBF (like LIF and NGAL) when combined with FGF-2 and TGF-α induces continued expansion of metanephric mesenchymes and their differentiation into organotypic epithelia within 7 days of organ culture (E-J). Tubules stain positive for E-cadherin (F), while glomerular-like structures express podocalyxin (Podxl) (G). Histologically, these structures resemble kidney epithelia at and beyond the S-shaped body stage (H-J). The sequence of metanephric mesenchymal differentiation in organ culture recapitulates epithelial differentiation in vivo (K; in this case in the presence of LIF). Arrows delineate a mesenchymal aggregate (after 3-4 days of differentiation in vitro) reminiscent of pretubular aggregates in vivo. UB, ureteric bud; CM, condensed mesenchyme; PA, pretubular aggregate; SB, S-shaped body; Tb, tubule; Gl, glomerular-like structure.

FIG. 21: β-catenin signaling triggers survival and proliferation of epithelial progenitors, but not tubulogenesis. Introduction of stabilized β-catenin (Ad-CTNNB_{S37A}) into epithelial progenitors marked by Pax-2 prevents apoptosis determined by immunostaining for activated caspase-3 (a-CASP3) observed after 3 days of culture under control conditions (Ad-GFP only). This anti-apoptotic response is blocked by dominant-negative TCF (Ad-DN-TCF).

### DETAILED DESCRIPTION OF THE INVENTION

The patent and scientific literature referred to herein establishes knowledge that is available to those skilled in the art. The issued patents, applications, and other publications that are cited herein are hereby incorporated by reference to the same extent as if each was specifically and individually indicated to be incorporated by reference.

There is a connection between factors secreted from the kidney that regulate nephron epithelium re-formation in adult kidney disease and mesenchyme induction into nephron epithelium in the embryonic kidney. Ureteric bud cells are known to secrete factors that stimulate the development, growth, conversion or differentiation of the metanephric mesenchyme into nephron epithelium. U.S. Pat. No. 6,432,681 identifies one of these factors as leukemia inhibitory factor (LIF) and describes the use of purified LIF, in combination with growth factors, to induce the formation of kidney epithelia from isolated metanephric tissue (See Barasch et al., Cell 99:377-386 (1999) and Yang et al., Dev Biol 246:296-310 (2002)).

Another factor specifically expressed in the ureteric bud is Ngal, an iron transporter that participates in the conversion of metanephric tissue to nephron epithelium by increasing cellular iron uptake (Yang et al., Mol Cell 10:1045-1056 (2002); Yang et al., Am J Physiol Renal Physiol 285:F9-F18 (2003); Li et al., Am J Physiol Cell Physiol 287:C1547-C1559 (2004)). Expression of Ngal plays a role in the morphogenesis of nephron epithelium by promoting the organization of cells into tubular structures, while suppression of Ngal expression by short hairpin RNA results in increased cyst formation by tubular cells (Gwira et al., J Biol Chem 280:7875-7882 (2005)). In the adult kidney, Ngal is the most highly overexpressed molecule in animal models of and humans with ischemic or nephrotoxic acute tubular necrosis (ATN) (J Am Soc Nephrol 14:2534-43 (2003)). Accordingly, U.S. Patent Application Publication No. US 2004/0219603 describes a method and kit for detecting the early onset of a renal tubular cell injury, including an ischemic renal injury and a nephrotoxic injury, by detecting Ngal as a biomarker in urine. In a mouse model of cisplatin-induced nephrotoxic injury, urinary excretion of Ngal increased within 3 hours of cisplatin injection, compared to 96 hours for detectable increases in conventionally measured biomarkers (Mischra et al., Am J Nephrol 24:307-315 (2004)). Intravenous administration of Ngal was found to be protective in a mouse model of a severe type of renal failure, ischemia-reperfusion injury (Mischra et al., J Am Soc Nephrol 15:3073-3082 (2004)). When a single dose of Ngal is administered during the initial phase of ischemia-reperfusion injury, the kidney is significantly protected (Mori et al., J Clin Invest 115: 610-621 (2005)).

Factors Secreted by the Ureteric Bud

Microarray technology was used to identify other ureteric bud factors that function as organ activators to regulate the differentiation or growth of metanephric tissue to nephron epithelium, and thus may be effective in protecting adult kidneys from damage or treating damaged kidneys. A group of molecules was identified as being expressed in the developing kidney and secreted from the ureteric bud (See Example 9). The group includes stem cell factor (also referred to herein as SCF, kit ligand, kitl), cytokine like factor-1 (also referred to herein as CLF-1, CLF1, CLF), CXCL14, FRAS1, neuropeptide Y, semaphorin 3C, Cyr 61, USAG-1 (also referred to herein as ectodin), IGF-BP2, WNT 6, WNT 9B, SHH, BMP-7, SOSTDC1, semaphorin 4D, NME3, laminin gamma 2, laminin alpha 5, laminin gamma 1, collagen triple helix repeat containing 1, nephronectin, collagen XVIII (also referred to herein as Col18a1), and laminin alpha 1. It is a discovery of the invention that stem cell factor, cytokine like factor-1, CXCL14, neuropeptide Y, semaphorin 3C, Cyr61, IGFBP2, semaphorin 4D, NME3 and collagen triple helix repeat containing 1 play a role in kidney development.

In one aspect, a composition is provided for modulating growth of metanephric tissue, the composition consisting essentially of two or more compounds selected from the group consisting of stem cell factor, cytokine like factor-1, CXCL14, FRAS1, neuropeptide Y, semaphorin 3C, Cyr 61, USAG-1, IGF-BP2, WNT 6, WNT 9B, SHH, BMP-7, SOSTDC1, semaphorin 4D, NME3, laminin gamma 2, laminin alpha 5, laminin gamma 1, collagen triple helix repeat containing 1, nephronectin, collagen XVIII and laminin alpha 1. Modulating the conversion of metanephric tissue to nephron epithelium using molecules endogenous to the developing kidney, specifically the ureteric bud, increases the probability that appropriate regulation of gene expression will be achieved.

In one aspect, a composition is provided for modulating growth of metanephric tissue, the composition consisting essentially of two or more compounds selected from the group consisting of stem cell factor, cytokine like factor-1, CXCL14, neuropeptide Y, semaphorin 3C, Cyr61, IGFBP2, semaphorin 4D, NME3 and collagen triple helix repeat containing 1.

In one aspect, a composition is provided for modulating growth of metanephric tissue, the composition consisting essentially of BMP-7 and cytokine like factor-1 (CLF-1).

In one aspect, a composition is provided for modulating growth of metanephric tissue, the composition consisting essentially of cytokine like factor-1 (CLF-1) and cardiotrophin like cytokine (CLC).

In one aspect, the compound comprises a purified polypeptide or fragment thereof. Methods for obtaining purified polypeptides or peptide fragments thereof are well known to those skilled in the art. Nonlimiting examples of such methods include de novo chemical synthesis, recombinant DNA technology and biochemical methods (i.e., chromatographic purification of polypeptides from cell or tissue extracts, or conditioned culture media).

For example, protein sequences are available for the human isoforms of stem cell factor (Accession No. AAA85450), cytokine like factor-1 (Accession No. AAC28335), CXCL14 (Accession No. AAD03839), FRAS1 (Accession No. AAH52281), neuropeptide Y (Accession No. AAA59944), semaphorin 3C (Accession No. AAH30690), Cyr 61 (Accession No. AAB58319), USAG-1 (Accession No. AAQ83296), IGFBP2 Accession No. AAA03246), WNT 6 (Accession No. AAD41674), WNT 9B (Accession No. AAQ88584), SHH (Accession No. AAA62179), BMP-7 (Accession No. AAH04248), SOSTDC1 (Accession No. Q6X4U4), semaphorin 4D (Accession No. AAH5450), NME3 (Accession No. AAH00250), laminin gamma 2 (Accession No. AAC50457), laminin alpha 5 (Accession No. AAH03355), laminin gamma 1 (Accession No. AAA59489), collagen triple helix repeat containing 1 (Accession No. AAH14245), collagen XVIII (Accession No. AAC39658), and laminin alpha 1 (Accession No. AAH39051). For example, the sequence is available for the murine isoform of nephronectin (Accession No. AAK84391).

Further provided for is a composition for modulating growth of metanephric tissue, the composition consisting essentially of WNT 6 and one or more compounds selected from the group consisting of stem cell factor, cytokine like factor-1, CXCL14, FRAS1, neuropeptide Y, semaphorin 3C, Cyr 61, USAG-1, IGFBP2, WNT 9B, SHH, BMP-7, SOSTDC1, semaphorin 4D, NME3, laminin gamma 2, laminin alpha 5, laminin gamma 1, collagen triple helix repeat containing 1, nephronectin, collagen XVIII and laminin alpha 1.

In one aspect, a composition is provided for modulating growth of metanephric tissue, the composition consisting essentially of WNT 9B and one or more compounds selected from the group consisting of stem cell factor, cytokine like factor-1, CXCL14, FRAS1, neuropeptide Y, semaphorin 3C, Cyr 61, USAG-1, IGFBP2, WNT 6, SHH, BMP-7, SOSTDC1, semaphorin 4D, NME3, laminin gamma 2, laminin alpha 5, laminin gamma 1, collagen triple helix repeat containing 1, nephronectin, collagen XVIII and laminin alpha 1.

In one aspect, a compound of the composition is an activator of the transcriptional complex β-catenin/TCF/Lef. Signaling pathways regulated by this transcriptional complex are involved in the regulation and survival of epithelial progenitor cells (see Example 11).

Ex Vivo Tissue Engineering and Transplant

After injury, the adult kidney displays an anatomical and functional recovery of renal integrity during which damaged nephrons are replaced by well-functioning nephrons. The reparative nature of the kidney indicates that the mesenchyme-to-epithelium conversion at the core of embryonic kidney development continues to be active in the adult kidney. Thus, it is important to identify conversion-inducing factors released from the ureteric bud during embryonic kidney development. One or more of the factors may be used as treatments to stimulate nephron repair after injury to the adult kidney, or to provide tissue engineering methods where kidneys, or components thereof, are engineered ex vivo from, for example, embryonic stem cells, adult stem cells, metanephric stem cells (embryonic stem cells contained in the metanephric mesenchyme), or renal tissue containing stem cells or metanephric stem cells.

A possible mechanism underlying nephron regeneration is the existence of adult (somatic) stem cells in the kidney which expand and differentiate in response to changes in the extracellular environment induced by the onset of injury or pathological conditions. Another possible mechanism is transdifferentiation, or interconversion, of differentiated renal cells into another renal cell type. In response to injury, the nephron epithelium has also been demonstrated to dedifferentiate into an active proliferative state characterized by the reappearance of mesenchymal markers detectable during nephrogenesis (for a review, see Anglani et al., J Cell Mol Med 8:474-487 (2004)).

The metanephric mesenchyme contains embryonic renal stem cells that give rise to epithelial cells, smooth muscle cells and endothelial cells (Oliver et al., Am J Physiol Renal Physiol 283:F799-F809 (2002)). Adult kidney stem cells have been localized in the renal papilla (Oliver et al., J Clin Invest 114:795-804 (2004)). Bone marrow stem cells may also repopulate the nephron after kidney injury. Human mesenchymal stem cells found in adult bone marrow can differentiate and contribute to functional complexes of a new kidney when the cells are implanted into a developing mouse embryo in culture followed by organ culture of the metanephric tissue isolated from the embryo (Yokoo et al., Proc Natl Acad Sci USA 102:3296-3300 (2005)).

Methods for growing organs ex vivo should avoid the use of xenogenic systems which can trigger the host immune system and lead to organ rejection following transplant. In one aspect, the methods of the present invention provide for the ex vivo growth of kidneys or kidney components by treating renal stem cells or kidney tissue (preferably autologous renal stem cells or autologous kidney tissue) with a cocktail of factors secreted endogenously by the ureteric bud, thereby inducing the formation of nephrons. Generating kidneys or components of kidneys from renal stem cells or kidney tissue under defined conditions decreases the risk of host rejection of the kidney upon transplant.

In one aspect, the invention provides for methods to facilitate ex vivo tissue engineering of kidneys or kidney components, followed by transplant of the engineered tissue into a subject suffering from kidney damage. In one aspect, the invention provides a method for modulating growth of embryonic or adult kidney tissue, the method comprising contacting the embryonic or adult kidney tissue with an effective amount of cytokine like factor-1. In another aspect, the invention provides a method for modulating growth of embryonic or adult kidney tissue, the method comprising contacting the embryonic or adult kidney tissue with an effective amount of stem cell factor. In another aspect, the invention provides a method for modulating growth of embryonic or adult kidney tissue, the method comprising contacting the embryonic or adult kidney tissue with an effective amount of any one of the compositions provided by the invention. In another aspect, the invention provides a method for modulating the conversion of metanephric tissue into nephron epithelium, the method comprising contacting the metanephric tissue with an effective amount of any one of the compositions provided by the invention.

The compositions provided by the invention can contact embryonic or kidney tissue in a plurality of settings to modulate the growth of embryonic or adult kidney tissue. In one embodiment of the invention, the tissue is in the kidney of a subject. In another embodiment, the tissue is isolated from an embryonic kidney, fetal kidney, developing kidney or adult kidney. In a further embodiment, after the tissue contacts the compound, the tissue is transplanted into a subject. In another embodiment, the subject is suffering from kidney damage. In other embodiments, the subject is suffering from or undergoing acute tubular necrosis, acute renal failure, transplant, diabetes, infection, surgery, ischemia, muscle damage, liver disease, blood transfusion, exposure to nephrotoxic medication or agents, or any combination thereof.

In one embodiment, the metanephric tissue is contained within an embryonic kidney, fetal kidney, developing kidney or adult kidney wherein the kidney is in an organ culture. In another embodiment, after the kidney contacts a composition of the invention, the kidney is transplanted into a subject. In further embodiments, the subject is suffering from or undergoing acute tubular necrosis, acute renal failure, transplant, diabetes, infection, surgery, ischemia, muscle damage, liver disease, blood transfusion, exposure to nephrotoxic medication or agents, or any combination thereof.

Methods for isolation, organ culture and transplantion of kidneys and metanephric tissue are described in U.S. Patent Application Publication Nos. US 2003/0086909 and US 2004/0191228, European Application No. 0 853 942, Hammerman, Am J Physiol Renal Physiol 283:F601-F606 (2002), Kanwar et al., J Clin Invest 98: 2478-2488 (1996), and Liu et al., Dev Biol 178:133-48 (1996).

In one embodiment, the metanephric tissue is contacted by the one or more compounds for a period of from about 24 hours to more than one day. In other embodiments, the methanephric tissue is contacted by one or more compounds for a period of less than about 24 hours, from about 24 to about 48 hours, from about 48 hours to about 3 days, from about 3 days to about 5 days, from about 5 days to about 10 days, from about 10 days to about 25 days, or for more than 25 days. Metanephric tissue is may be treated for over 48 hours with one compound or a combination of compounds. Adult kidney is may be treated with one or more compounds in a single dose.

In another embodiment, the effective amount of the composition comprises from about 50 nanograms to about 50 micrograms. In another embodiment, the effective amount of each compound in the composition comprises from about 50 nanograms to about 50 micrograms. For each compound, the amounts used in treating embryonic kidney tissue or adult kidney tissue may be greater than the naturally occurring amounts. For example, kit ligand can be used at an effective concentration of about 500 nanograms/milliliter and CLF-1/CLC can be used at an effective concentration of about 3 nanomolar.

Exemplary methods for determining the conversion of metanephric tissue into nephron epithelia are described in U.S. Pat. No. 6,423,681. Microscopy may be used to visualize enlargement of the metanephric tissue and the formation of tubules. Biochemical techniques, such as immunohistochemistry, can be used to determine if the metanephric tissue develops characteristics of nephron precursors. E-cadherin and collagen IV exhibit unique expression patterns during nephrogenesis in both in vitro and in vivo settings. Immunolocalization of E-cadherin and collagen IV may then be used to demonstrate the conversion of metanephric tissue to nephron precursors.

In Vivo Methods for Treating Kidney Damage

During kidney organogenesis, Ngal is expressed in the ureteric bud and participates in the differentiation of metanephric tissue to nephron epithelium. In the adult kidney, Ngal participates in protecting the kidney tissue from damage. Using the dichotomous function of Ngal as a model, the ureteric bud factors of the present invention may be assessed for their ability to protect the developed kidney from damage and/or to repair damaged kidney tissue (i.e., reformation of functional nephron epithelium). Non-limiting examples of methods for evaluating the use of the ureteric bud factors as therapeutic compounds are discussed in Example 10 using Ngal as an exemplary ureteric bud factor.

In one aspect, a method is provided for treating damaged kidney tissue, the method comprising administering to a subject an effective amount of a composition provided for by the invention. In one embodiment, the subject is suffering from kidney damage resulting from acute tubular necrosis, acute renal failure, transplant, diabetes, infection, surgery, ischemia, muscle damage, liver disease, blood transfusion, exposure to nephrotoxic medication or agents, or any combination thereof.

In another embodiment, the administering comprises intralesional, intraperitoneal, intramuscular or intravenous injection; infusion; liposome-mediated delivery; or topical, nasal, oral, ocular or otic delivery. In one embodiment, the administering is through the renal artery.

In other embodiments, the compositions of the invention can be administered to a subject for the purpose of treating kidney damage, rescuing the kidney from damage, or prophylactically for any operation or testing that induces kidney damage or acute renal failure.

Kits for Treating Kidney Damage

In one aspect, a kit is provided for treating kidney damage comprising one or more of the compositions provided by the invention in an effective amount to modulate growth of embryonic or adult kidney tissue. In one embodiment, the kit is used during dialysis.

In another embodiment, the kit is used with a drug delivery pump. In an additional embodiment, the pump is connected by a catheter to the renal artery. In another embodiment, the pump is implanted into a subject. Implantable drug delivery pump/catheter systems may be used for continuous, site-specific infusion of a composition into the kidney via the renal artery. Direct administration of a composition to the kidney may be accomplished by adapting implantable drug delivery pump/catheter systems such as those described in U.S. Pat. Nos. 5,643,207 and 6,283,949. The composition is dispensed through a catheter from a subcutaneously implanted pump comprising a reservoir. The catheter is implanted into a preferred site of the organ of interest, in this case the renal artery of the kidney. These systems provide for controlled local administration of a composition to the organ. The commercially-available SynchroMed® and IsoMed® Infusion systems manufactured and sold by Medtronic, are currently used for direct infusion of chemotherapeutic agents into the liver through the hepatic artery. Canine models of renal transplant have adapted similar implantable pump/catheter systems for direct infusion of drugs into the kidney through the renal artery (Gruber et al., J Surg Res 71:137-144 (1997); Gruber et al., Transplantation 53:12-19 (1992); Gruber et al., J Pharmacol Exp Ther 252:733-738 (1990)).

Another embodiment encompasses using the kit to prepare embryonic kidney cells, adult kidney cells, or a combination thereof for use in a bioartificial hemofiltration device. In another embodiment, the bioartificial hemofiltration device is implanted into a subject. U.S. Pat. Nos. 5,549,674, 5,686,289 and 6,150,164, and U.S. Patent Application Publication Nos. US2001/0041363 and US2003/0119184 are directed toward methods and compositions of a bioartificial kidney suitable for use in vivo or ex vivo. The bioartificial kidney comprises living renal tubule cells seeded along the surface of a perfused hollow fiber bioreactor to reproduce the ultrafiltration function and transport function of the kidney. The bioartificial kidney has been used successfully in Phase I/II clinical trials (Humes et al., Kidney Int 66:1578-1588 (2004)).

Embryonic kidney cells or adult kidney cells, preferably metanephric cells or adult renal stem cells, can be prepared for use in the bioartificial kidney by pretreatment with the kit of the present invention. Because the compositions contained in the kit facilitate the formation of nephron epithelium in vivo, pretreatment with the compounds will provide for the physiologically-accurate formation of nephron epithelium directly on the surface of the semipermeable hollow fiber contained in the bioartificial filtration device.

Terms

In one aspect of the invention, the pharmacologically active agent or composition can be combined with a carrier. The term “carrier” is used herein to refer to a pharmaceutically acceptable vehicle for a pharmacologically active agent. The carrier facilitates delivery of the active agent to the target site without terminating the function of the agent. Non-limiting examples of suitable forms of the carrier include solutions, creams, gels, gel emulsions, jellies, pastes, lotions, salves, sprays, ointments, powders, solid admixtures, aerosols, emulsions (e.g., water in oil or oil in water), gel aqueous solutions, aqueous solutions, suspensions, liniments, tinctures, and patches suitable for topical administration.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of ≦20%.

The term “effective” is used herein to indicate that the inhibitor is administered in an amount and at an interval that results in the desired treatment or improvement in the disorder or condition being treated (e.g., an amount effective to modulate the growth of kidney tissue).

In some embodiments, the subject is a human, mouse, rabbit, monkey, rat, bovine, pig, sheep, goat, cow or dog.

Pharmaceutical formulations include those suitable for oral or parenteral (including intramuscular, subcutaneous and intravenous) administration. Forms suitable for parenteral administration also include forms suitable for administration by inhalation or insufflation or for nasal, or topical (including buccal, rectal, vaginal and sublingual) administration. The formulations may, where appropriate, be conveniently presented in discrete unit dosage forms and may be prepared by any of the methods well known in the art of pharmacy. Such methods include the step of bringing into association the active compound with liquid carriers, solid matrices, semi-solid carriers, finely divided solid carriers or combinations thereof, and then, if necessary, shaping the product into the desired delivery system.

The following examples illustrate the present invention, and are set forth to aid in the understanding of the invention, and should not be construed to limit in any way the scope of the invention as defined in the claims which follow thereafter.

### EXAMPLES

### Example 1

### Function of FGFs and TIMPs in Conversion and Maintenance of Mesenchymal Cells

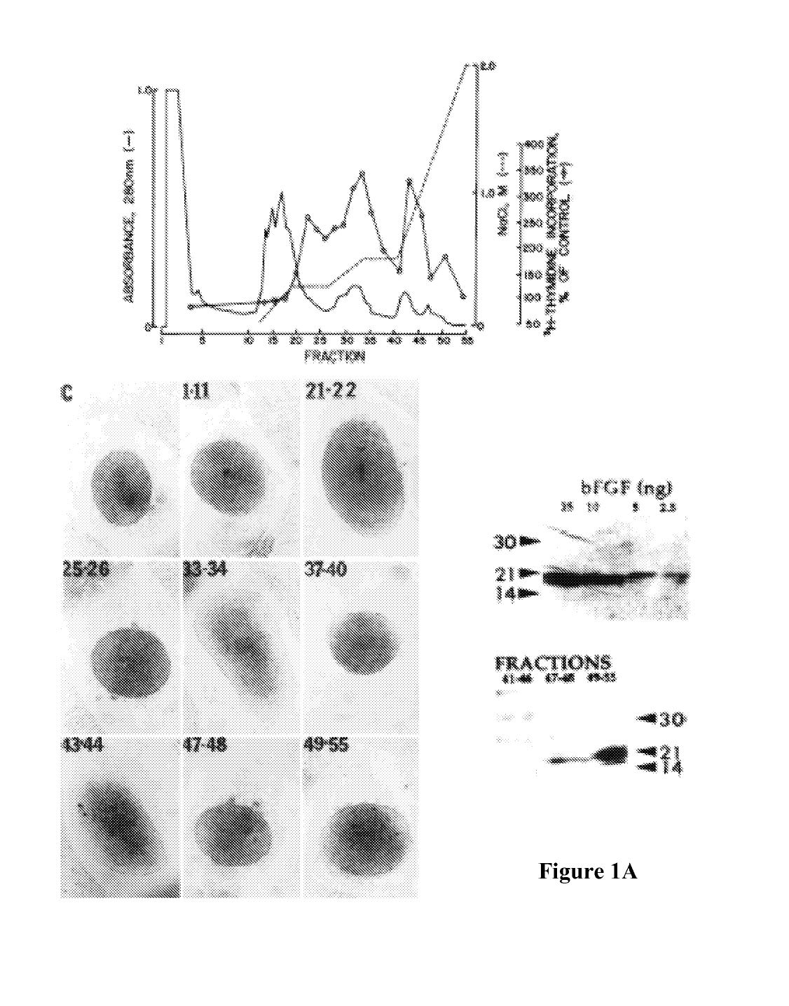

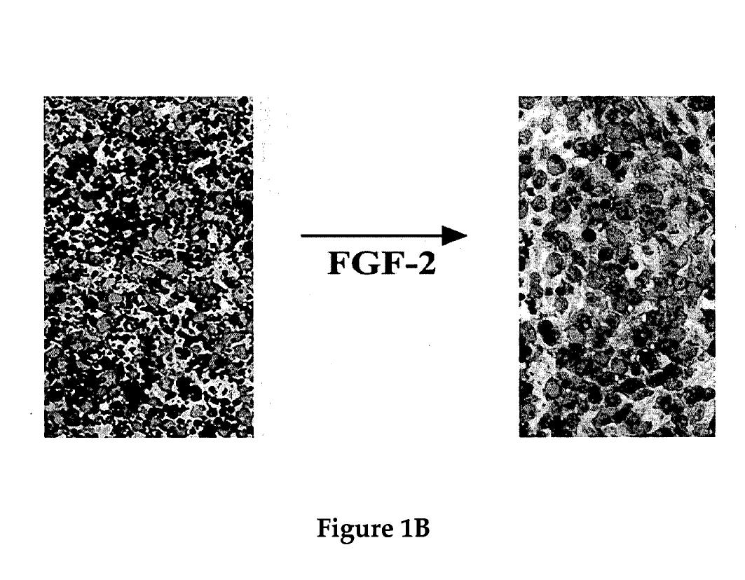

Metanephric mesenchyme dies when separated from the ureteric bud, but it undergoes extensive growth for days-weeks in culture when treated with UB proteins. This allows the mesenchyme to be cultured in serum free conditions. By protein purification, FGF-2 and FGF-9 were identified as active stimuli (FIGS. 1A-1B). Screening ureteric bud RNA with Affymetrix gene chips also showed that FGF-18 is specific to the ureteric bud and induces growth of the mesenchyme (Sakurai et al., Proc Natl Acad Sci USA 94:6279-6284 (1997)). Co-expression of these FGFs is common to other organs.

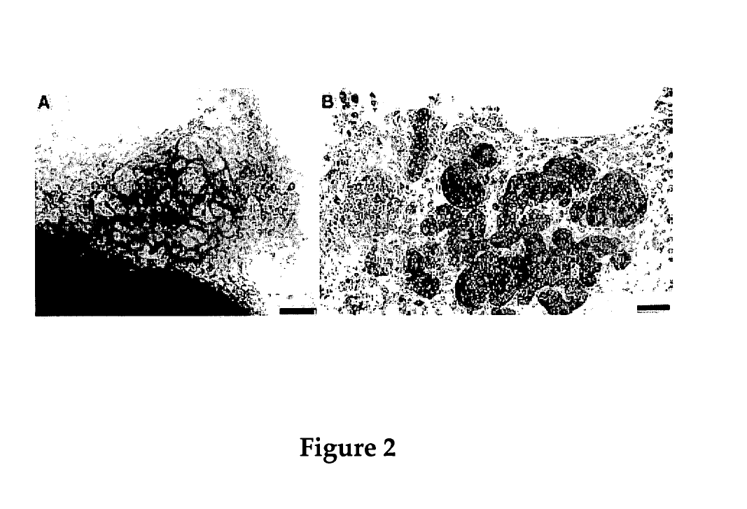

To determine if FGFs stimulate cell conversion, metanephric mesenchyme was grown for a number of days with FGF-2 but cells did not convert to epithelium, even using RT-PCR to assay for epithelial proteins (Barasch et al., Am J Physiol 271(1 Pt 2):F50-F61 (1996)). To determine if FGF-2-grown mesenchyme contained competent epithelial progenitors, inducing tissue was added and it was found that mesenchymal cells maintained in FGF-2 for 4-7 days could still form epithelia (FIG. 2). Mesenchyme without FGF-2, in contrast, underwent cell death. FGF-9 and -18 were also permissive as to the formation of epithelia. These experiments revealed that while the FGFs do not trigger epithelialization, they permitted the survival of mesenchymal progenitors (Barasch et al., Am J Physiol (Renal Physiol 42):F757-F767 (1997)). Additionally, the data showed that induction of epithelia and the proliferation of progenitors are regulated independently of one another.

Similar studies were performed after isolation and sequencing of a second family of growth factors from UB cells, the tissue inhibitors of metalloproteinases (TIMP; see Corcoran et al., 1995; Seo et al, Cell 114: 171-180). As demonstrated by RT-PCR, in situ hybridization and reverse zymography, TIMP-2 and TIMP-3 were expressed by the UB, although TIMP-2 protein was the most abundant member of the family (Barasch et al., J Clin Invest 103:1299-1307 (1999)). Because TIMP-2 not only stimulated mesenchymal growth but also inhibited UB branching, TIMPs may coordinate the growth of the mesenchyme and the ureteric bud.

### Example 2

### Mesenchymal to Epithelial Conversion in rat Metanephros is Induced by LIF

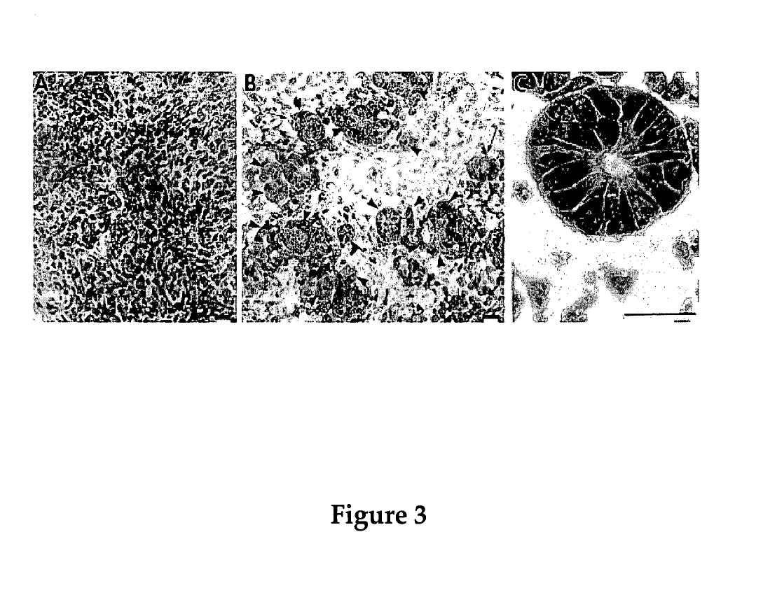

In experiments where spinal cord fragments are used as a source of metanephric-inducing molecules, epithelialization requires a ‘second’ stimulus to induce mesenchymal conversion. To identify whether the ureteric bud produces this second signal, rat mesenchyme was incubated with FGF-2 and then added UB proteins (Barasch et al., Cell 99:377-386 (1999)). The combination induced hundreds of cysts, tubules and early nephrons (FIG. 3). The conversion was reproduced in every mesenchyme (n>1000), and was visible to the naked eye, allowing rapid screening of chromatographic fractions. Leukemia inhibitory factor (LIF) was purified as the kidney inducer. Recombinant LIF was also inductive; its inductive activity was synergistic with TGFβ2.

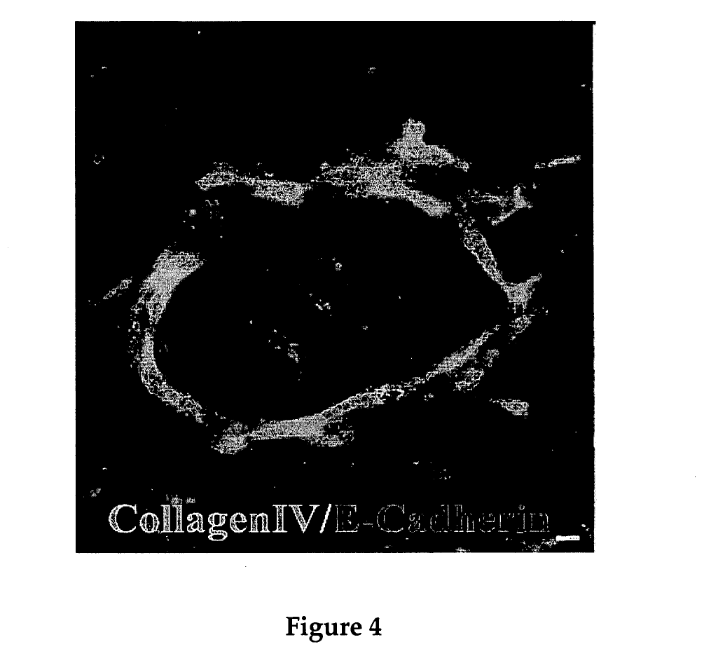

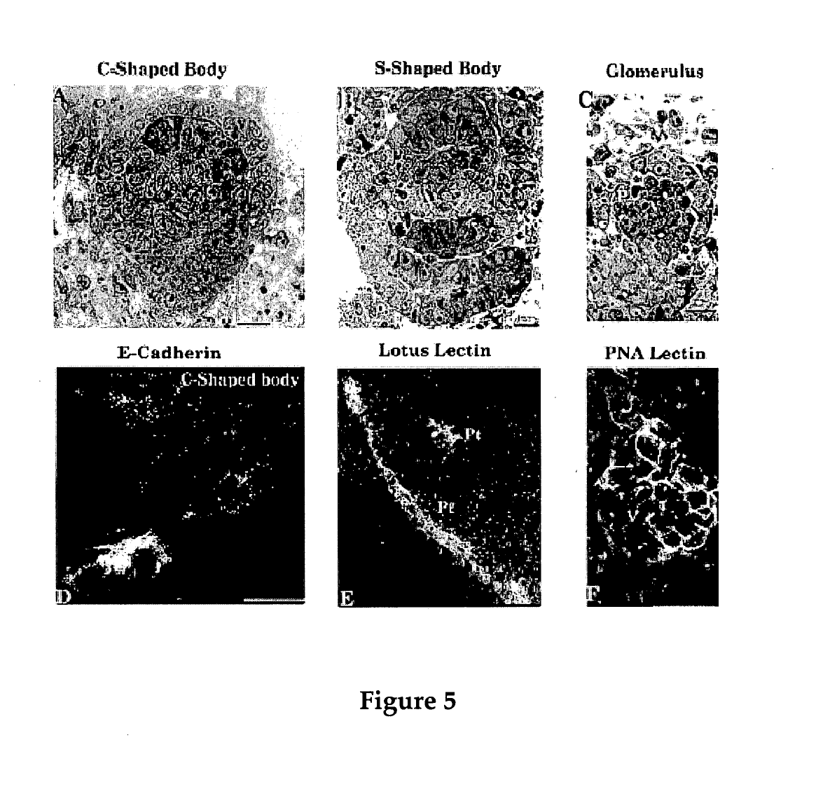

The tubules produced by LIF were characterized. There were many C-shaped bodies (early nephron precursors) which expressed E-cadherin at one pole (the future distal convoluted tubule) and cadherin-6 in proximal segments, a pattern typical of the early nephrons. A basement membrane of collagen IV—an induced epithelial protein—surrounded the bodies (FIG. 4). There were also many advanced S-shaped bodies with glomeruli and tubules, and prominent expression of Lotus Lectin, a proximal tubule marker (FIG. 5). Contamination of the ureteric bud was excluded as causative, by the absence of UB proteins, and by repeating the cultures with E_{11.5} rat mesenchyme, which is present before the ureteric bud has actually formed. These studies demonstrate the induction of differentiated nephrons.

To determine whether the action of LIF was consistent with the classical definition of kidney induction, LIF was withdrawn after only 24 hrs of incubation, but this did not block tubulogenesis. In contrast, withdrawal of FGF-2 led to apoptosis, indicating that FGF, unlike LIF, did not convert the cell type. Hence, induction by LIF is reminiscent of fragments of spinal cord used in classical experiments.

IL-6 Family and Receptors: RT-PCR and gene chip analysis were used to locate the LIF receptor (a heterodimer of LIFR and gp130). LIF receptor and gp130 were readily detectable in metanephric mesenchyme while LIF was specific to ureteric bud. Other IL-6 cytokines were also detected in the bud, and these were also inductive.

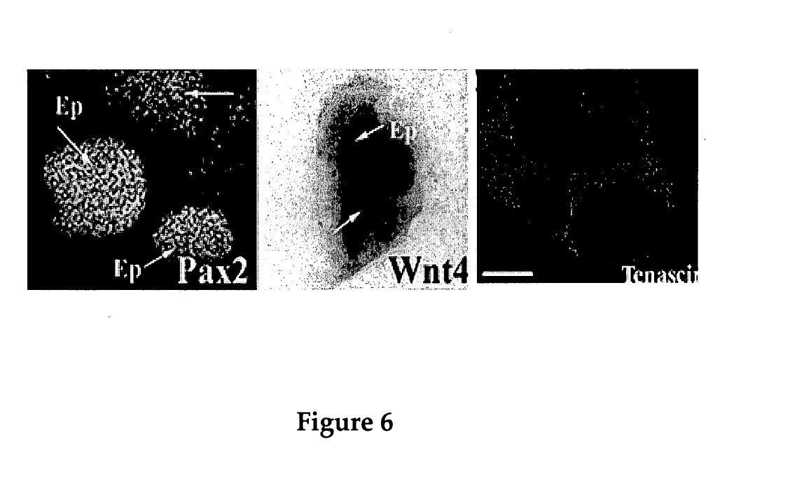

LIF and Growth Factors: LIF required a growth factor (FGF>EGF>TIMP) to induce epithelia; alone it had no activity. This showed that LIF targeted mesenchymal cells that were maintained, expanded, or made competent to respond to LIF by the other factor. To test this further, freshly dissected mesenchyme treated with FGF-2 (>8 hours) was examined and clusters of cells were found which expressed Pax-2, WT-1, and Wnt-4, but not tenascin, a stromal marker (FIG. 6). LIF stimulated the appearance of the second messenger, phospho-Y⁷⁰⁵-STAT-3 in these clusters, but not in the rest of the tissue, indicating that LIF acted on these progenitors, and these clustered cells were competent to form epithelia. Hence, FGF maintained competent epithelial progenitors that were then targeted by LIF. A developmental role for LIF and its related cytokines has been described in neurons, astrocytes, and hematopoeitic stem cells.

### Example 3

### An Epithelial Precursor is Regulated by the Ureteric Bud and by the Renal Stroma

The clusters of Pax-2, WT-1, and Wnt-4 cells never expressed epithelial proteins (even after prolonged culture), but within 24 hours of adding LIF, they expressed many epithelial proteins such as E-cadherin, ZO-1 and lamininα₅ (Yang et al., Dev Biol 246:296-310 (2002)). Over a 4-day period, these cells aggregated and formed tubules and nephrons. This sequence of events showed that LIF targeted late staged mesenchymal cells (i.e. Wnt-4⁺ cells) triggering the expression of epithelial proteins.

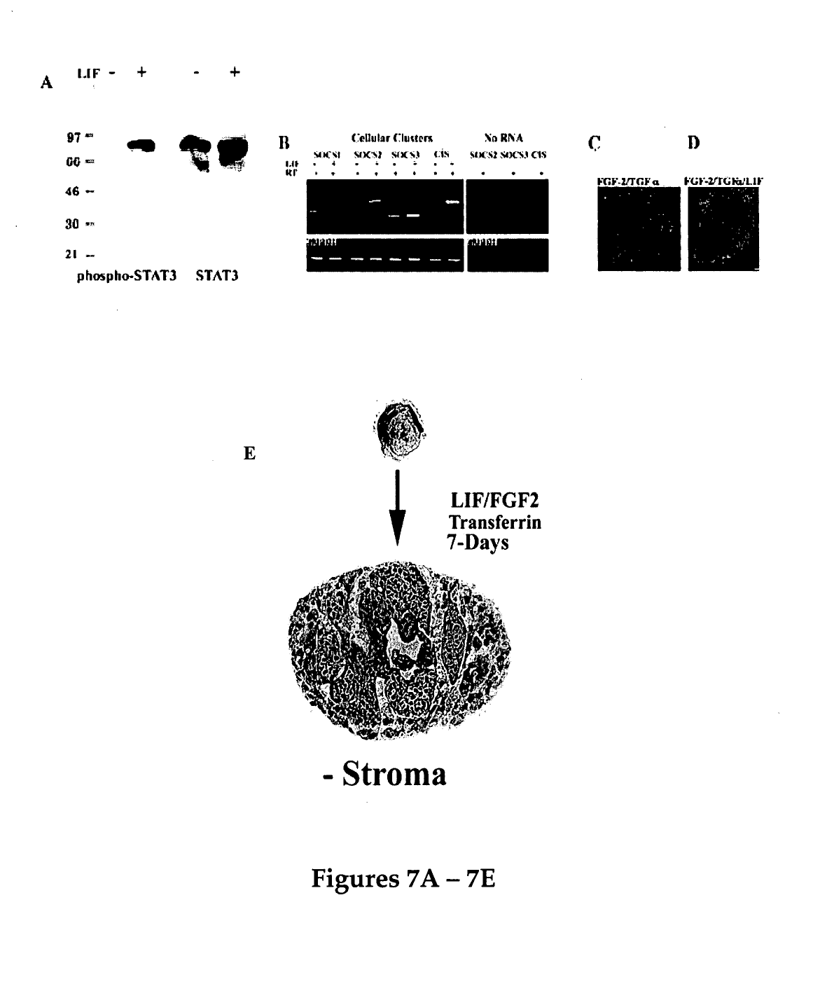

To determine whether the Pax-2⁺, WT-1⁺, Wnt-4⁺ clusters could be induced in the absence of other types of mesenchymal cells, the clusters were isolated with a needle and LIF and FGF-2 were then added. This activated a variety of relevant second messengers (STAT-3, STAT dependent SOCS and CIS genes), followed by growth of these cells and glomerulo-tubulogenesis (FIGS. 7A-7E). This shows that signaling from other cell types is not necessary for LIF induction in vitro.

To determine if the target cells were multipotent progenitors or were already restricted to one fate, single cells were labeled in the Pax-2⁺, WT-1⁺, Wnt-4⁺ cluster using a clonal dilution of the LacZ-retrovirus. LIF induced these cells to produce both glomeruli and tubules. These experiments indicate that UB cytokines acted on uncommitted cells. Because 9.5±1.3 SEM (range 4-28; n=78) epithelial cells were produced from a single Wnt-4⁺ progenitor cell, it seems that LIF stimulated 2-5 cell cycles, during which time the cells assumed a mature phenotype. This response is reminiscent of the transit amplification of stem cells.

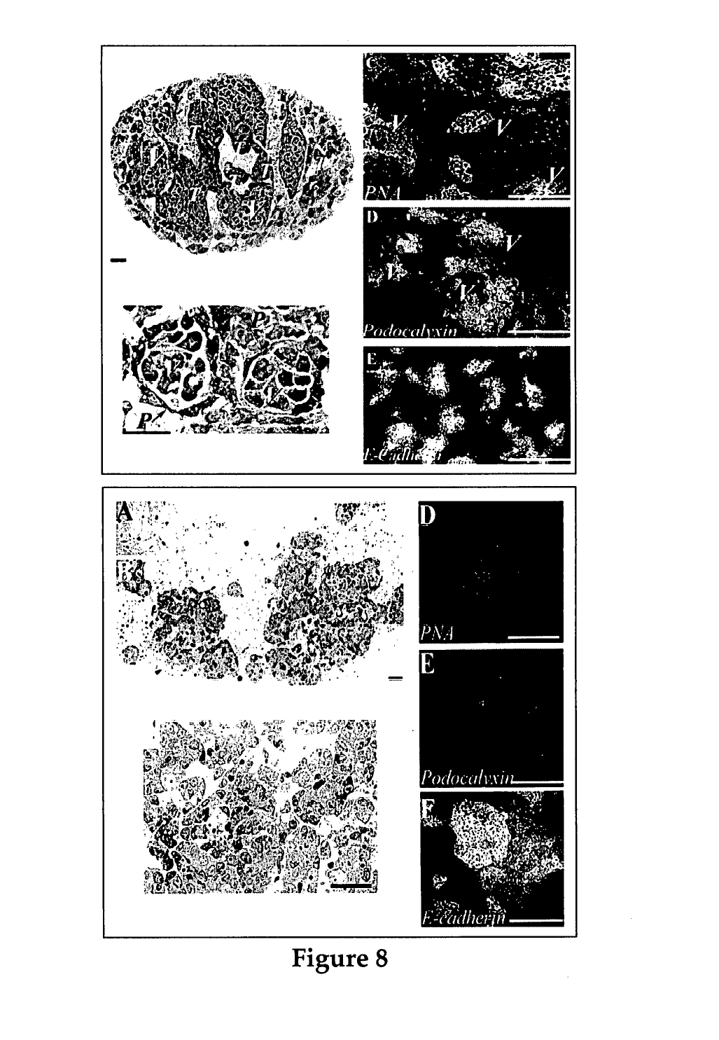

Pax-2⁺, WT-1⁺, Wnt-4⁺ cells are normally surrounded by stroma. To test signaling by these cells, BF-2⁺ cells or proteins from these cells were combined with the Pax-2⁺, WT-1⁺, Wnt-4⁺ clusters and found that the stroma inhibited LIF induction. This was demonstrated by a complete loss of glomerulogenesis (FIG. 8). Hence while the ureteric bud stimulates epithelial conversion, factors from renal stroma block epithelial conversion.

### Example 4

### Iron Delivery Pathway Mediated by a Lipocalin

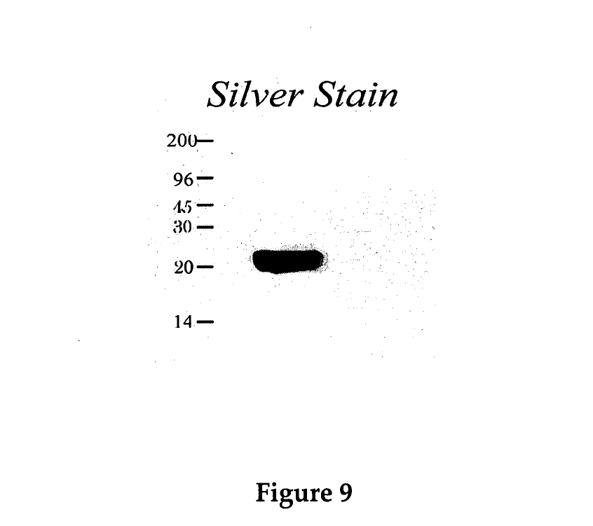

An inducer, called 24p3 or neutrophil-gelatinase associated lipocalin (Ngal), is specifically expressed by the ureteric bud and has been purified (FIG. 9) (Yang et al., Mol Cell 10:1045-1056 (2002)).

On the basis of crystallographic data of the molecule cloned in bacteria, Ngal is an iron transporter. The evidence that Ngal transports iron includes the purification of ⁵⁹Fe-Ngal from ⁵⁹Fe loaded UB cells by column chromatography and by immunoprecipitation. In addition, Ngal permitted growth of the embryonic kidney in the absence of transferrin, indicating that the Ngal could serve as an iron donor. The genetic response to Ngal also had many similarities with transferrin. Both proteins upregulated ferritin and downregulated transferrin receptor1 in cell lines, and they produced a strikingly similar global response in metanephric mesenchyme as detected by gene chip assays. In addition, when cloned Ngal was treated with reducing equivalents, low pH, and then gallium (a metal that occupies iron binding sites in proteins and siderophores (Cui et al., Dev Dyn 226:512-522 (2003); Ward et al., Inorg Chem 38:5007-5017 (1999)) but cannot undergo redox reaction), induction of nephrons was blocked. These data, combined with the crystallographic data from recombinant Ngal, demonstrates that Ngal traffics iron, and that iron transport is necessary for nephron induction by Ngal.

Delivery of iron classically involves endocytosis of a carrier into acid vesicles, where iron is released to a divalent metal::proton synporter (DMT1) for export to the cytoplasm. To determine if Ngal also delivers iron by an endocytic pathway, fluorescent-Ngal and fluorescent-transferrin were used. Both of these molecules were endocytosed into cells, and at steady state, they overlapped only to a small degree.

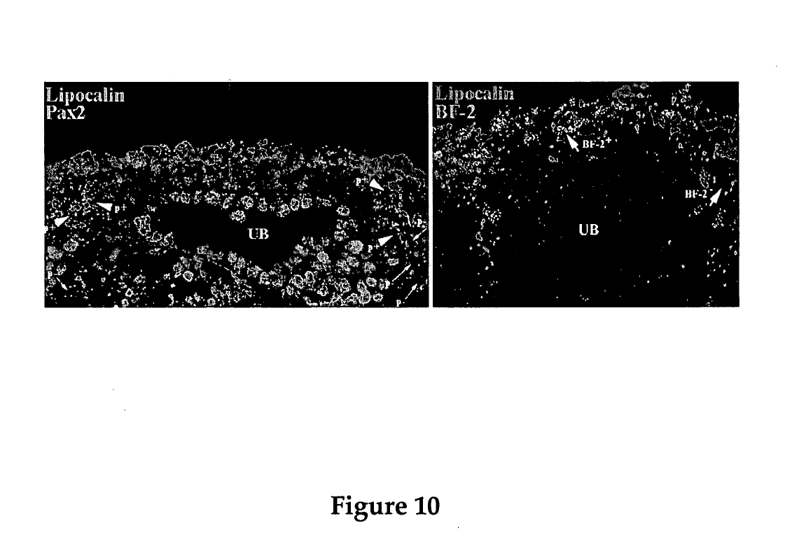

The targeting of Ngal and transferrin was even more divergent in embryonic kidney (FIG. 10). Ngal was taken up in the periphery by Pax-2⁺, Wnt-4⁻ cells and by BF-2⁺ stromal cells (detected by incubating BF-2-β-galactosidase expressing animals with fluorescent Ngal), whereas transferrin was strictly incorporated by some, but not all cells aligned with the ureteric bud. In situ assays for transferrin receptor1 demonstrated the location of the transferrin pathway to these late cells. Ngal stimulated proliferation of these peripheral cells as determined by the incorporation of BrdU, an activity that was not reproduced by transferrin. These data show the distinct targeting of Ngal and transferrin, and that iron delivery to different cell types and stages of kidney development is mediated by different iron transporters.

### Example 6

### Detection of Intracellular Iron Activity with a Genetic Probe

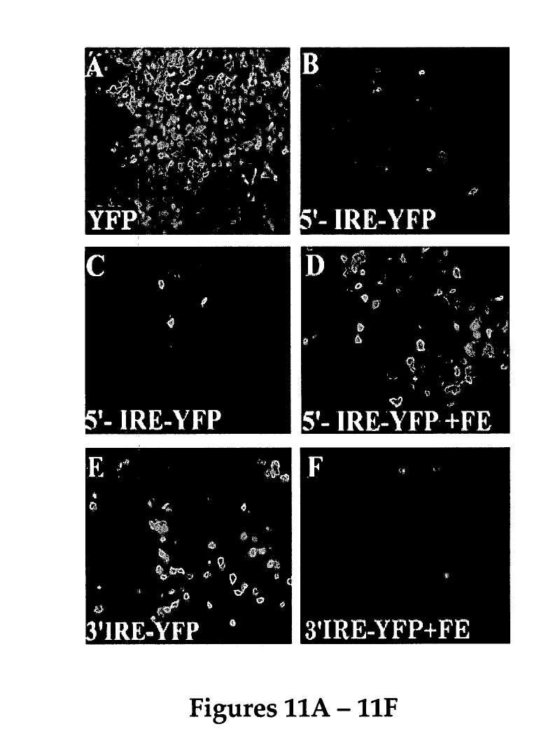

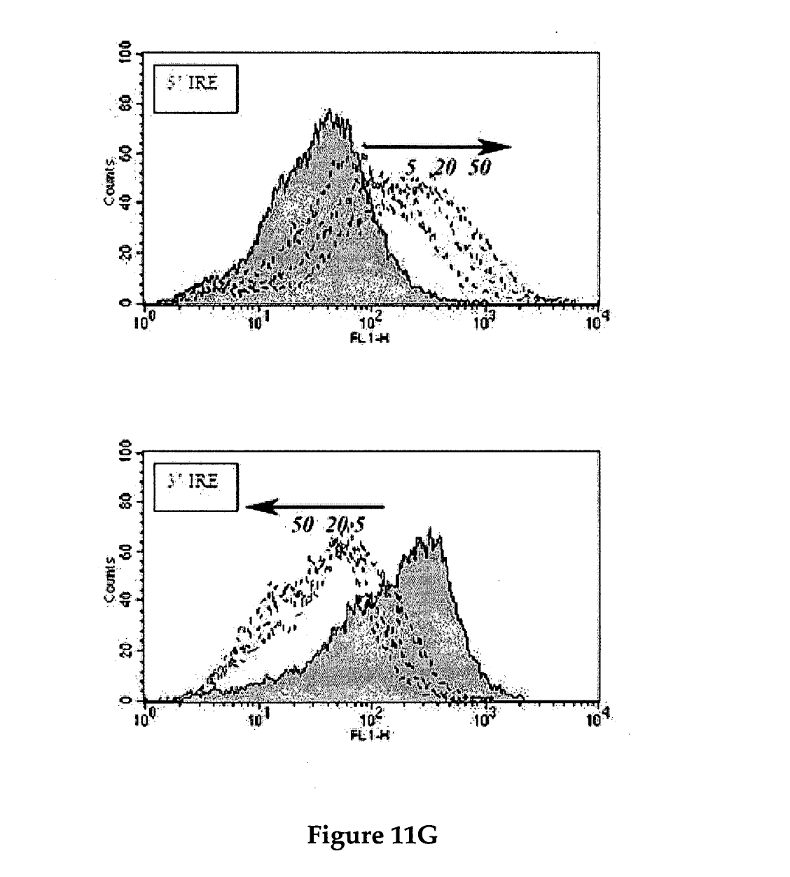

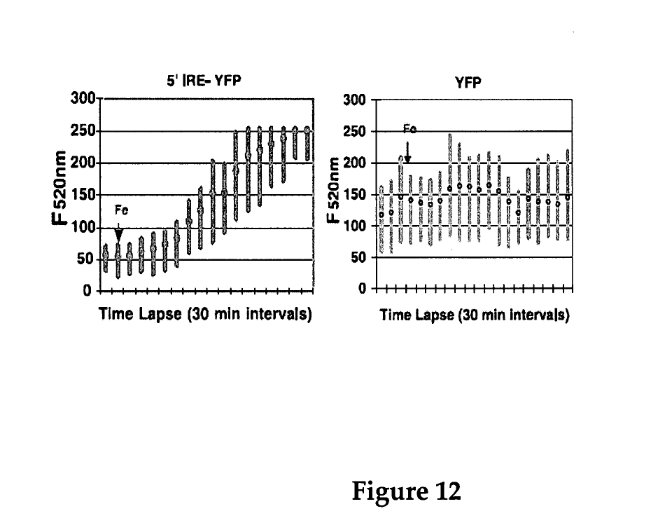

Two iron sensors have been developed to measure changes in the regulatory pool of iron during development (FIGS. 11A-11G) (Li et al., Am J Physiol Cell Physiol 287:C1547-C1559 (2004)). The 5′ Iron Response Element (IRE) of ferritin or the 3′Iron Response Element of transferrin receptor1 was ligated to destabilized fluorescent proteins and the probes were introduced into stable cell lines. Probes were tested by loading cells with iron carriers or alternatively removing iron with chelators, and then following the response by microscopy, immunoblot, FACS analysis and time-lapse photography with single-cell measurements. FIGS. 11A-11G show that 5′ IRE fluorescence increases with iron loading, and conversely 3′ IRE fluorescence decreases with iron loading. FACS analysis showed that these responses were dose dependent, and had a dynamic range of 10 fold, comparing 5′ IRE and 3′IRE. The change in fluorescence was time dependent and could be visualized in single cells after 30 min-1 hr (FIG. 12). Both 5′ and 3′ fluorescent changes were fully reversible, as shown by opposing responses to iron chelators and by the withdrawal of iron. Ngal also produced an iron dependent response.

### Example 7

### Identification of Ngal a Biomarker for Ischemic Renal Injury

Ngal not only induces embryonic cells but also rescues the adult kidney from cell death (Mischra et al., J Am Soc Nephrol 14:2534-2543 (2003)). Ngal is the most highly over-expressed molecule in many models of Acute Tubular Necrosis (ATN), including both ischemic and nephrotoxic ATN.

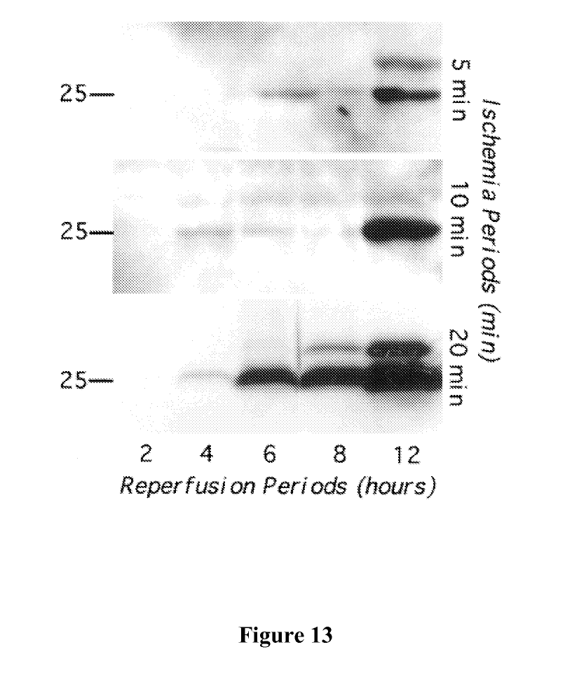

Ngal was the most over-expressed RNA (by microarray) in the proximal tubule of three animal models and humans with ATN. It was found in regenerating cells and in the urine after 3 hrs of ischemia. The greater the ischemic time in mouse kidney, the greater the production of Ngal (FIG. 13). ATP depletion in human proximal cells also induced Ngal, showing that damage to this epithelia resulted in Ngal expression. Human kidney samples show strong expression of Ngal in cells damaged by ATN. The reason for this expression pattern is to recover iron and maintain the adult epithelial phenotype.

### Example 8

### Ngal Rescues the Kidney from ATN

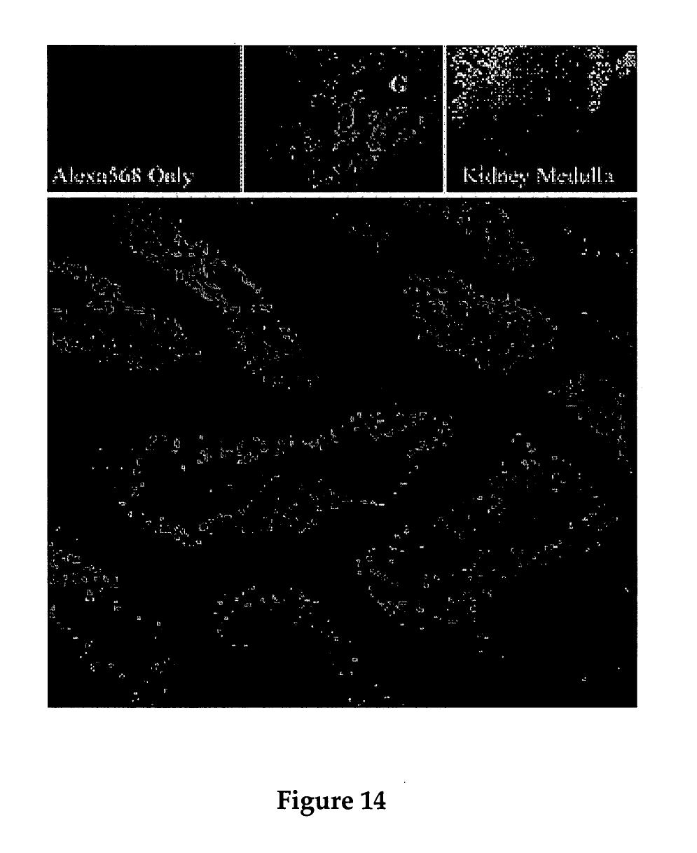

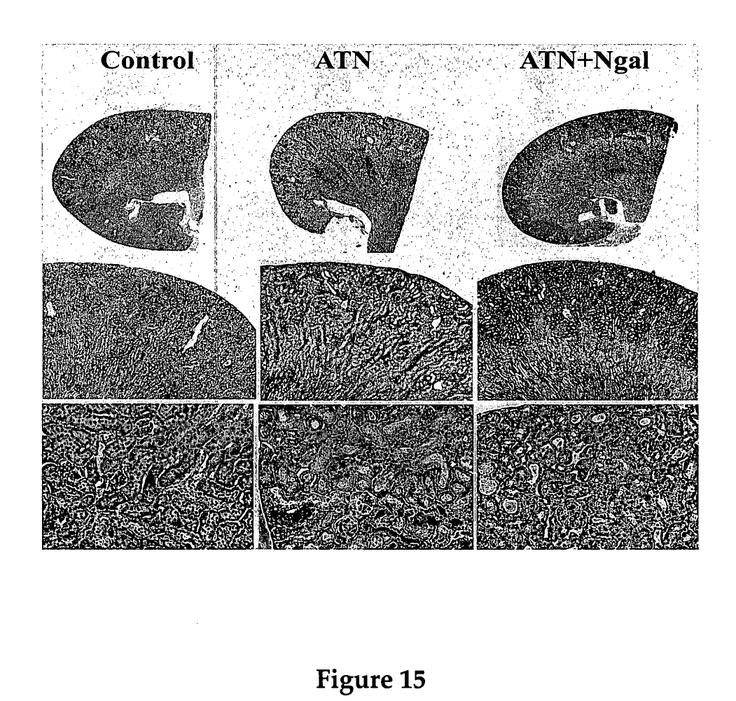

To determine if expression of Ngal is protective or destructive after insult to the proximal tubule, Ngal was labeled with a fluorescent molecule and found to be filtered by the glomerulus and taken up by the proximal tubule (FIG. 14). A single injection of Ngal at the time of, or within one hour of renal ischemia, blocked ATN (Mori et al., J Clin Invest 115:610-621 (2005)). This was demonstrated by a lower Creatinine level (Cr=3.2±0.2 vs Cr=1.1±0.2 p<1.8×10⁻⁵) and by rescued histology on the biopsy of the kidney (FIG. 15).

### Example 9

### Identification of Genes that Regulate Nephrogenesis

Embryonic tissue from rat mesenchyme was used as a sensor for molecules secreted from ureteric bud cells. The activity of these molecules is measured as a growth response followed by the formation of nephrons. The protein that triggers these responses is then identified by growing thousands of flasks of ureteric bud cells, harvesting the media in which the cells are growing, and then fractionating this media by a process of chromatography. The starting material is first fractionated into 20 parts and each is assayed on 20 dissected metanephric mesenchymes (the nephron progenitor). This process is then repeated 4-5 times as the active fraction is serially processed through 4-5 columns. The benefit of using a bioassay is that it is a blinded ‘non-candidate gene approach’ that allows the discovery of unexpected molecules, including LIF, CLF and Ngal, which generate nephrons.

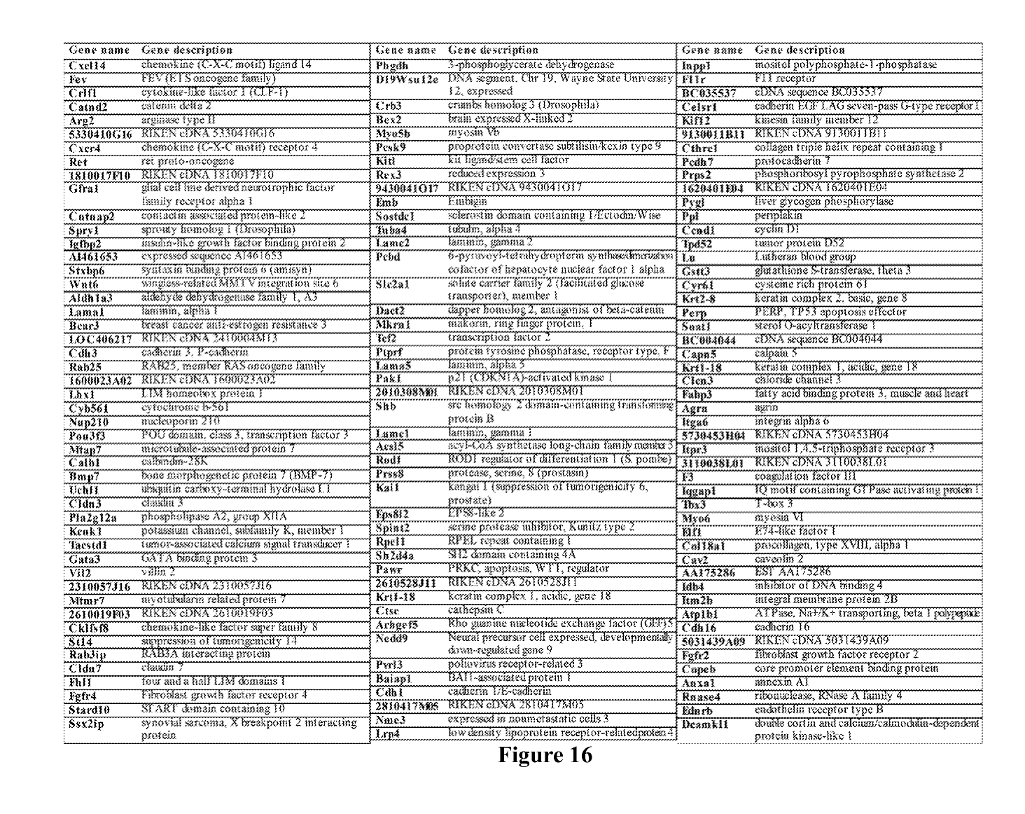

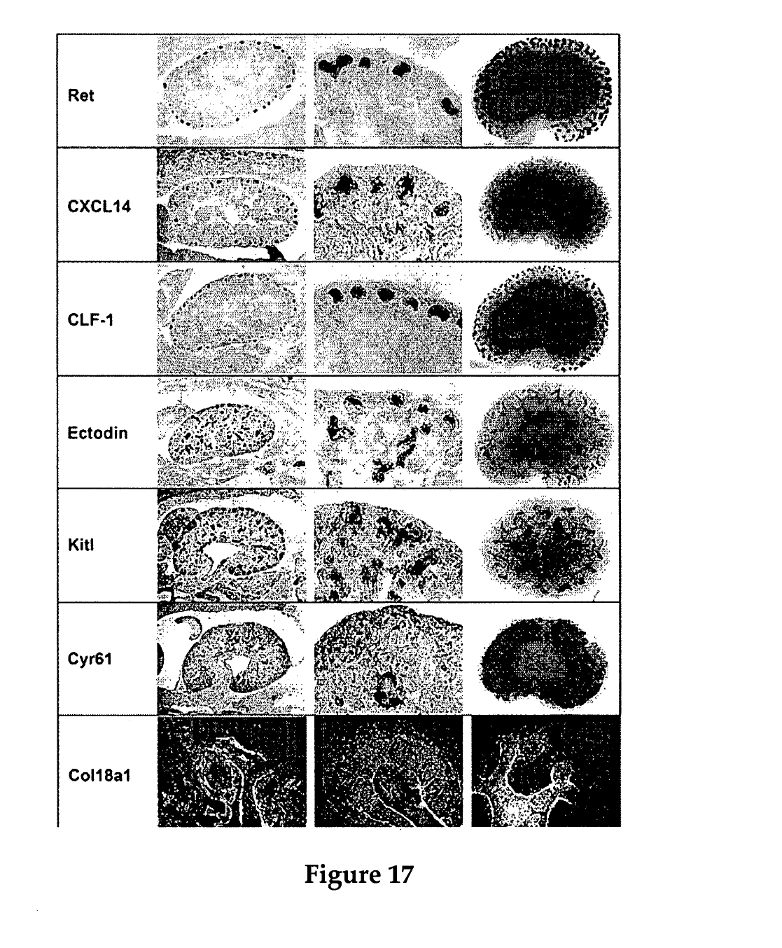

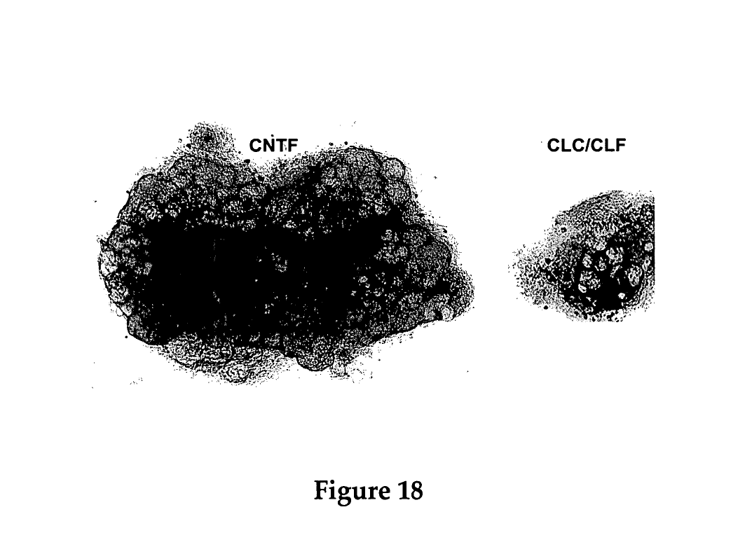

An alternative approach utilizing microarray technology was used to identify genes expressed by the ureteric bud. Using this technology, 605 molecules were identified that have least a 2-fold enrichment in the ureteric bud compared to rat metanephric mesenchyme. Of these molecules, 390 are known genes and 215 have yet to be described. Using in silico analyses that identify members of the secretonome (proteins that are secreted and can act as growth factors) and/or published accounts indicating secretion, 41 candidate growth genes were identified (FIG. 16) (Grimmond et al; Genome Res (2003) 13:1350). This list was compared to other published databases from the mouse and from the human (Dekel, Kidney Int (2003) 64:1588) to assay for evolutionary conservation of gene expression. A set of secreted molecules, which included SCF, CLF-1, CXCL14, FRAS1, Neuropeptide Y, Semaphorin 3c, Cyr 61, USAG-1, collagen XVIII and Ret, was analyzed further by RT-PCR and the presence of Ret, CXCL14, CLF-1, USAG-1 (ectodin), SCF (kitl), Cyr61 and collagen XVIII (Col18a1) in the developing kidney was then determined by in situ hybridization (FIG. 17). The invention provides for characterization of these molecules in the kidney.

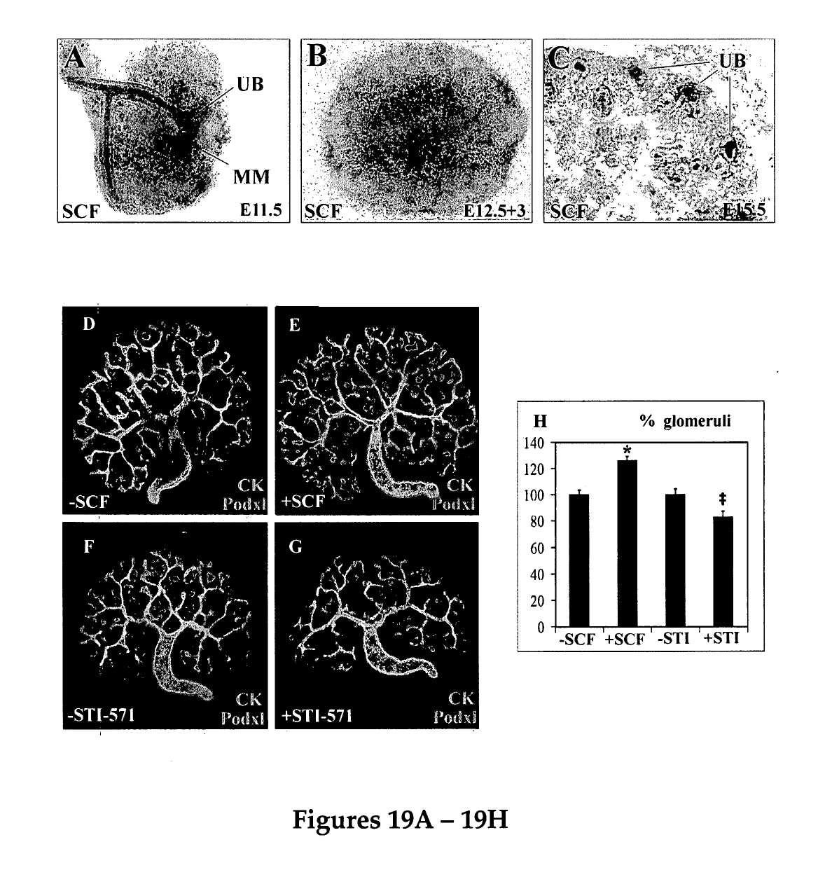

The factor with the highest relative enrichment in the ureteric bud is stem cell factor (SCF), a well-known survival and proliferation factor for primordial germ cells, melanoblasts, and hematopoietic precursors. SCF signals via the receptor tyrosine kinase c-kit and induces second messengers (STAT3) which are typical of LIF. SCF is expressed in the branching ureteric bud (FIGS. 19A-19H) (Nature 347:667, 1990).

Cytokine like factor-1 (CLF-1) forms a complex with cardiotrophin-like cytokine (CLC). CLC has not been shown to be expressed in the kidney. The heterodimeric cytokine complex interacts with the membrane-bound ciliary neurotrophic factor (CNTF) receptor and with gp130 and the LIF receptor, similar to the interaction of LIF with receptors. CLF-1/CLC activates the same signaling pathways as LIF. Knock-out of CLF results in a phenotype where mice are unable to suckle, but the kidney phenotype has not yet been investigated.

The small inducible cytokine CXCL14 belongs to the family of chemokines that are generally involved in immune responses. The family signals through 7 trans-membrane spanning G-protein coupled receptors. However, the membrane receptor for CXCL14 is currently unknown. CXCL14 is expressed in the adult proximal tubule, but the expression pattern is not known.

Fras1 is expressed in the duct system of the early urogenital system and it is essential for renal development because knockout of Fras1 leads to various kidney phenotypes ranging from renal agenesis to cystic dysplasia. This protein may function extracellularly because it contains a domain (ECM3) that is similar to a component of extracellular matrix in sea urchins. Mutations in the FRAS1 gene have recently been identified as causative in the development of Fraser syndrome, a congenital disorder affecting several systems including the kidney (McGregor et al., Nat Genet 34:203-208 (2003); for review see Yu et al., Curr Opin Genet Dev 14:550-7 (2004)). Fras1 expression has been demonstrated in the ureteric bud; and in Fras1 knockout mice, the UB forms and invades metanephric mesenchyme, but induction of the mesenchyme does not occur (Vrontou et al., Nat Genet 34:209-214 (2004)).

Neuropeptide Y (NPY) is an abundant and widespread peptide in the mammalian nervous system, where it stimulates proliferation. It acts also as a differentiation factor for neuronal precursors. Neither the kidney expression pattern of NPY nor the renal phenotype in NPY knock-outs have been investigated in detail. Because NPY is expressed in the ureteric bud, and its receptor is expressed in the metanephric mesenchyme and developing epithelia, NPY may mediate inductive signaling.

The semaphorins comprise a large family of phylogenetically conserved, secreted and transmembrane signaling proteins, which are known to guide the growth and migration of axons. Sema3C can act as either a repellent or an attractant for axons and for vascular cells in culture and mice with a Sema3C knockout have malformations of the aorta. Sema3C is not yet been described in the kidney.

Cyr61 is a secreted factor that is involved in the formation of blood vessels, possibly by binding integrins. However, the biology of Cry61 in the developing kidney has not been reported. Like Ngal, Cyr61 is strongly induced in ischemia-induced acute tubular necrosis of the kidney (Mischra et al., J Am Soc Nephrol 14:2534-2543 (2003); Muramatsu et al., Kidney Int 62:1601-1610 (2002)).

The proposed function of USAG-1 is to modify signaling by a secreted factor called Wnt, which is critical for epithelial development in the kidney. The data to date on USAG-1, however, is focused on the frog embryo, where manipulation of its level of expression changes cell fate in a manner consistent with a role in Wnt signaling (Yanagita et al., Biochem Biophys Res Comm 316:490-500 (2004)). The effect of USAG-1 depends on components of the canonical Wnt pathway including the Wnt co-receptor and the intracellular signaling molecule called β-catenin. USAG-1 has also been shown to act as an antagonist of bone morphogenic protein-7 (BMP-7; Yanagita et al., Biochem Biophys Res Comm 316:490-500 (2004). In embryonic kidneys, BMP-7 has been shown to be expressed in the ureteric bud, mesenchymal cell aggregates and developing nephrons (Yanagita et al., Biochem Biophys Res Comm 316:490-500 (2004)). USAG-1 expression in the developing kidney has not been reported and a genetic mutant is not currently available.

Harvesting the Candidate Regulators

To discern their function, identified proteins can be cloned and synthesized, then applied dissected metanephric mesenchyme. The genes can be evaluated for their ability to induce growth of the tissue, conversion of the mesenchyme into epithelia, or inhibitory effects. In addition, growth and branching of the ureteric bud can be used to elucidate the regulatory roles of the proteins. Subsequently, the proteins can be knocked-down in the growing kidney to determine whether development remains intact or is blocked. The knock-down can be accomplished using small interfering RNA (siRNA) wherein the mesenchyme is incubated with RNA to create unstable duplexes in the cell. This technique can be used in the developing kidney and it provides the flexibility to rapidly screen identified molecules to determine which are necessary for renal development. If a molecule is found to be active, and necessary for development, its activity can be determined in vivo by over-expressing it from the ureteric bud. This can be done by cloning the molecule into the HoxB-7 promoter, an expression cassette developed at Columbia and specific for expression from the ureteric bud (Srinivas et al., Dev Genet 24:241-251 (1999)).

Application to Human Models

There has been recent progress in understanding the developing kidney using mouse and rat models. To translate this data into human cells, cloned proteins can be analyzed in human kidneys to determine whether they activate renal growth and development. Small interfering RNA constructs can be used to determine whether the genes provided for by the invention are required for renal development. These studies will be important to identify the ultimate therapeutic target and establishing systematic methods to evaluate renal development.

### Example 10

### Ngal as a Drug for Renal Disease

Expression of Ngal in Damaged Kidney of Man and Mouse.

The expression pattern of Ngal with long periods of ischemia typical of clinical disease can be determined and the source of Ngal that appears in the urine with acute renal failure can be identified.

The expression of Ngal in kidneys subjected to different degrees of ischemic damage is measured first by immunoblots of blood and urine using affinity purified polyclonal anti-mouse and anti-human antibodies that were generated from rabbits using purified Ngal protein.

There are two variables in models of ischemic nephropathy (a) the length of time of the reduction of blood flow by cross-clamp (b) the length of time of reperfusion after removal of the crossclamp. The solution to this two variable model was developed, wherein a series of timed measurements during reperfusion are made in urine and blood for each degree of renal ischemia. This scheme allows one to examine blood and urine over a wide range of ischemic conditions and follow a time course of expression during recovery. Depending on these results, animals are selected at different stages of this ischemia-reperfusion injury and the kidneys are harvested for detection of Ngal expression. Preliminary experiments show that Ngal will be most highly expressed in the proximal tubule.

Cadaveric Renal Transplant Model of Renal Ischemia

During the harvesting and transport of the kidney, the proximal tubule undergoes ATN. This limits the utility of cadaveric kidneys, because patients must receive powerful immunosuppressants while awaiting recovery of renal function. Hence, a model of renal ischemia can be used that mimics transplant ATN by performing mouse to mouse transplants. Periods of warm and cold ischemia can be varied to mimic cadaveric transplants and Ngal expression is determined after the kidney is re-perfused in the recipient.

Screening Rat and Mouse Kidneys Subjected to Chemical Damage Including Aminoglycoside Antibiotics and Chemotherapeutic Agents

Initial work with cis-platinum-induced nephrotoxicity demonstrates that Ngal is expressed within 12 hours of administration. These experiments are clinically important and depict the potential breadth of function of Ngal in a large variety of renal diseases.

Screening Human Diseases for the Expression of Ngal

Human kidney expresses Ngal in the proximal tubule. The greater the cellular damage, the greater the expression of Ngal. Human archival material can be screened to detect the location of Ngal expression and to determine its specificity for ATN.

Screening Human Urine and Blood Samples for Ngal

Ngal is an early response marker of ATN in rodents. One can collect human urine and blood samples for screening Ngal expression in human samples. A variety of types of ATN can be analyzed including ischemic, nephrotoxic and post-cadaveric transplant nephropathy.

Ngal Rescues Rodent Kidney from ATN

Many prior studies establish the time course of ATN in animal models including renal cross clamp and transplant ischemic damage. Using these models Ngal can be administered as a continuous infusion and as a single dose.

The Schedule of Dosing of Ngal In Vivo

Administration before, during and within 1 hour of the onset of renal ischemia or chemical damage uses this protein as a protective agent. Administration after ischemic damage examines the potential of Ngal in epithelial repair. Initial studies show that a single dose of Ngal (100 micrograms) before or 1 hour after the renal artery cross-clamp causes renal protection.

Dose of Ngal

The NGAL receptor has not been identified and Ngal concentrates in the proximal tubule (raising its local concentration). Hence a series of experiments are required to determine the minimal dosage required to rescue the kidney in vivo (See Mori et al., J Clin Invest 115(3):610-621 (2005)). This is an important variable because it impacts on the potential toxicity of the protein, if present.

Production of Ngal

Ngal is a binding protein for siderophores, which are small proteins produced by bacteria in order to chelate iron. Ngal protein can be produced in a form containing the bacterial siderophore and in a form that lacks this molecule. Both of these reagents can be introduced into animals with ischemic kidney damage. In addition, a mouse model that overproduces Ngal from liver cells can be produced by introducing a potent adenovirus that carries the gene for Ngal. The adenoviral-mouse will synthesize and secrete Ngal directly from liver cells in animals that will be subjected to renal damage. This obviates the need to use bacterially expressed recombinant Ngal, and the virus is expected to provide protection against ischemia.

Determination of Ngal Toxicity

To determine potential toxicity, Ngal is injected on a daily basis for a number of weeks and then any resulting changes in cell populations are assessed throughout the body. Proliferative and apoptotic indices are measured by use of BrDU labeling and Apo-tag kits. Given that only a single injection of Ngal is required, and the protein is produced endogenously, toxicity is not expected.

Mechanism of Action of Ngal

Uptake of Ngal

The receptor for Ngal is suggested to be megalin, a promiscuous molecule at the luminal side of the proximal tubule. The urine from a megalin knockout animal contains an abundance of Ngal. These data can be confirmed by infusing the commercially available Receptor Associated Protein (RAP) which serves as an endogenous inhibitor of megalin. RAP should block the uptake of fluorescent Ngal, and if uptake into the proximal tubule is essential for its biological activity, then RAP should block Ngal mediated rescue of the proximal tubule. This experiment also demonstrates that luminal receptors such as megalin (facing the urinary space) are important for the effect of Ngal, whereas putative basolateral receptors (facing the blood side) are not likely to contribute to the delivery of Ngal to the proximal tubule.

Cell Trafficking

In cells of the embryonic kidney, Ngal enters vesicles that are distinct from canonical pathways such as the transferrin recycling pathway and the lysosomal pathway. If trafficking of Ngal in the adult kidney is mediated by megalin, however, then Ngal will be targeted to lysosomes. If Ngal traffics to non-lysosomal compartments, then it may be reutilized by recycling from the cell. If targeted to lysosomes, then it is destroyed after a single pass.

Second Messenger Signaling

Ngal is a carrier for iron. Hence it serves to chelate iron, directing it from dying cells to regenerative cells. This supplies iron and in addition blocks the toxicity of free iron released from dying cells. In addition, more typical methods of cellular signaling can be examined. To this end, embryonic kidney was screened for dozens of signaling pathways using pathway specific antibodies and anti-phosphorylation antibodies and imaging gels (Molecular Probes). A pathway dedicated to Ngal in the embryonic kidney would then be replicated in the adult tubule. However, despite screening with over 30 antibodies to detect activation of standard signaling pathways, not were identified. Thus, Ngal serves primarily as an iron carrier.

Application to Human Models

Rescue of adult human kidneys from acute tubular necrosis can be demonstrated by showing that treatment with Ngal activates the same signaling pathway (such as the accretion of iron) in human kidneys as in mouse. This can be done using discarded human kidneys purchased commercially. Further, using these kidneys, the preservation of the proximal tubule after exposure to Ngal can be examined. Isolation of proximal tubule is a common protocol and can be demonstrated initially with mouse kidneys.

### Example 11

### Activators of the Transcriptional Complex b-catenin/TCF/Lef Rescue from Apoptosis Kidney Progenitor Cells

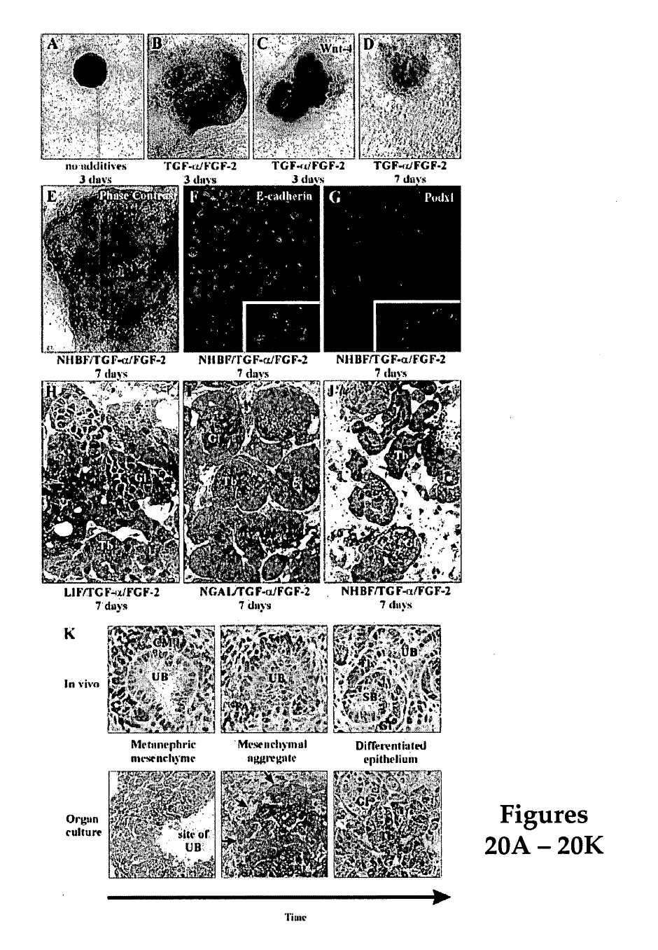

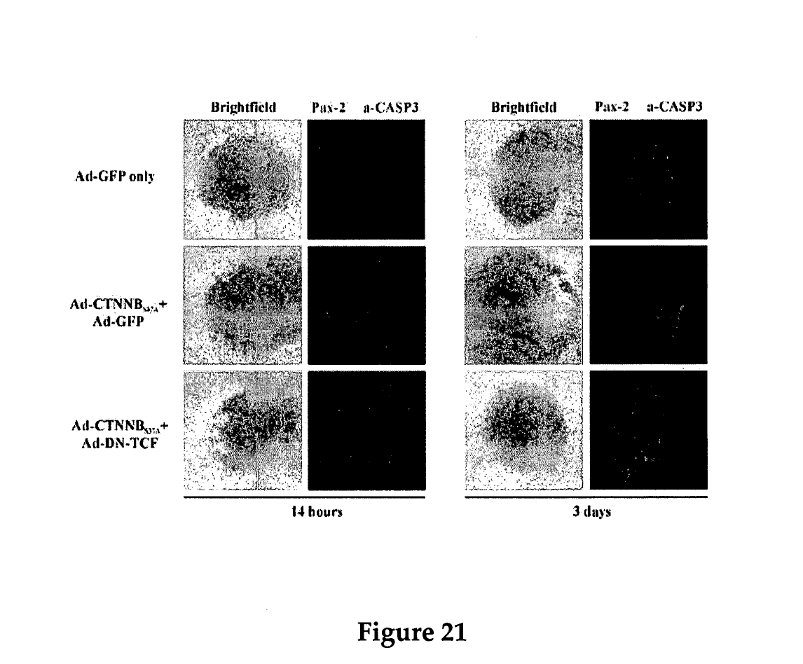

In the absence of stimulation by exogenous Wnt ligands, epithelial differentiation of metanephric mesenchyme is characterized by an activation of multiple TCF/Lef-dependent targets of β-catenin. β-catenin/TCF/Lef signaling is involved in the regulation of survival and proliferation of epithelial progenitor cells and induces stage progression characterized by an induction of a subset of the tubulogenic transcriptional program. Cells with impaired TCF/Lef-dependent transcription are progressively depleted during epithelial differentiation, indicating that this signaling pathway con control cellularity in the renal epithelial lineage. See FIGS. 20A-20K and 21.

While the foregoing invention has been described in some detail for purposes of clarity and understanding, these particular embodiments and examples are to be considered as illustrative and not restrictive. It will be appreciated by one skilled in the art from a reading of this disclosure that various changes in form and detail can be made without departing from the true scope of the invention.

What is claimed is:

1. A composition for modulating growth of metanephric tissue, the composition consisting essentially of two or more compounds selected from the group consisting of stem cell factor, cytokine like factor-1, CXCL14, FRAS1, neuropeptide Y, semaphorin 3C, Cyr 61, USAG-1, IGFBP2, WNT 6, WNT 9B, SHH, BMP7, SOSTDC1, semaphorin 4D, NME3, laminin gamma 2, laminin alpha 5, laminin gamma 1, collagen triple helix repeat containing 1, nephronectin, collagen XVIII, and laminin alpha 1.

2. The composition of claim 1, wherein one selected compound is WNT 6.

3. The composition of claim 1, wherein one selected compound is WNT 9B.

4. A composition for modulating growth of metanephric tissue, the composition consisting essentially of two or more compounds selected from the group consisting of stem cell factor, cytokine like factor-1, CXCL14, neuropeptide Y, semaphorin 3C, Cyr 61, IGFBP2, semaphorin 4D, NME3 and collagen triple helix repeat containing 1.

5. A composition for modulating growth of metanephric tissue, the composition consisting essentially of cytokine like factor-1 and BMP7.

6. A composition for modulating growth of metanephric tissue, the composition consisting essentially of cytokine like factor-1 and cardiotrophin like cytokine.

7. A method for modulating growth of embryonic or adult kidney tissue, the method comprising contacting the embryonic or adult kidney tissue with an effective amount of cytokine like factor-1.

8. A method for modulating growth of embryonic or adult kidney tissue, the method comprising contacting the embryonic or adult kidney tissue with an effective amount of stem cell factor.

9. A method for modulating growth of embryonic or adult kidney tissue, the method comprising contacting the embryonic or adult kidney tissue with an effective amount of the composition of claim 1.

10. The method of claim 9, wherein growth of embryonic or adult kidney tissue comprises conversion of metanephric tissue to nephron epithelium.

11. The method of claim 9, wherein the tissue comprises metanephric stem cells, renal stem cells, or both.

12. The method of claim 9, wherein the tissue is in the kidney of a subject.

13. The method of claim 9, wherein the tissue is isolated from an embryonic kidney, fetal kidney, developing kidney, or adult kidney.

14. The method of claim 9, wherein the tissue is contained within an embryonic kidney, fetal kidney, developing kidney, or adult kidney, wherein the kidney is in an organ culture.

15. The method of claim 13, wherein the tissue is transplanted into a subject.

16. The method of claim 13, wherein the kidney is transplanted into a subject.

17. The method of claim 12, wherein the subject is a human.

18. The method of claim 12, wherein the subject is suffering from kidney disease or kidney damage.

19. The method of claim 12, wherein the subject is suffering from or undergoing acute tubular necrosis, acute renal failure, transplant, diabetes, infection, surgery, ischemia, muscle damage, liver disease, blood transfusion, exposure to nephrotoxic medication or agents, or any combination thereof.

20. The method of claim 9, wherein the effective amount of the composition comprises from about 50 nanograms to about 50 micrograms.

21. A method for treating damaged kidney tissue, the method comprising administering to a subject an effective amount of the composition of claim 1.

22. The method of claim 21, wherein the subject is suffering from kidney damage resulting from acute tubular necrosis, acute renal failure, transplant, diabetes, infection, surgery, ischemia, muscle damage, liver disease, blood transfusion, exposure to nephrotoxic medication or agents, or any combination thereof.

23. The method of claim 21, wherein the administering comprises intralesional, intraperitoneal, intramuscular or intravenous injection; infusion; liposome-mediated delivery; or topical, nasal, oral, ocular or otic delivery.

24. The method of claim 23, wherein the administering is through the renal artery.

25. The method of claim 23, wherein the effective amount of each compound in the composition comprises from about 50 nanograms to about 50 micrograms.

26. A kit for treating kidney damage comprising the composition of claim 1 in an effective amount to modulate the growth of embryonic or adult kidney tissue.

27. The kit of claim 26, wherein the kit is used during dialysis.

28. The kit of claim 26, wherein the kit is used with a drug delivery pump.

29. The kit of claim 26, wherein the pump is connected by a catheter to the renal artery.

30. The kit of claim 26, wherein the pump is implanted into a subject.

31. The kit of claim 26, wherein the kit is used to prepare embryonic kidney cells, adult kidney cells, or a combination thereof for use in a bioartificial hemofiltration device.

32. The kit of claim 31, wherein the bioartificial hemofiltration device is implanted into a subject.

33. A method for protecting a kidney from damage, the method comprising contacting the kidney with an effective amount of the composition of claim 1.

34. A method for stimulating nephron repair, the method comprising contacting the nephron in need of repair with an effective amount of the composition of claim 1.
